(19) United States
(12) Patent Application Publication
(10) Pub. No.: US 2002/0110850 A1
(43) Pub. Date: Aug. 15, 2002

(54) Human genes, sequences and expression products-16

(76) Inventors: Craig A. Rosen, Laytonsville, MD (US); Steven M. Ruben, Olney, MD (US); Patrick J. Dillon, Gaithersburg, MD (US); Haodong Li, Gaithersburg, MD (US); William A. Haseltine, Washington, DC (US)

Correspondence Address: HUMAN GENOME SCIENCES INC, 9410 KEY WEST AVENUE, ROCKVILLE, MD, 20850

(21) Appl. No.: 09/783,590

(22) Filed: Feb. 15, 2001

Related U.S. Application Data
(63) Continuation of application No. 08/420,856, filed on Apr. 12, 1995, now abandoned.

(51) Int. Cl. C12Q 1/68; C07H 21/04; C12N 9/00; C12P 21/02; C12N 5/06; C07K 14/435

(52) U.S. Cl. 435/69.1; 435/325; 435/183; 435/320.1; 530/350; 536/23.2; 435/6

(57) ABSTRACT

A DNA sequence of SEQ ID NOS:1-12483. An isolated DNA sequence containing the coding region of a human gene and a DNA sequence identified in SEQ ID NOS:1-12483. An isolated DNA sequence containing the coding region of a human gene that contains a DNA sequence present in ATCC Deposit No. 75916. A DNA sequence hybridizable with a DNA sequence of SEQ ID NOS:1-12483 and isolatable from other DNA in ATCC Deposit No. 75916. Expression vectors containing any of the above. Proteins expressed from any of the above.

This invention relates to newly identified polynucleotide sequences corresponding to transcription products of human genes, and to complete gene sequences associated therewith and to expression products thereof as well as to uses for the foregoing.

Identification and sequencing of human genes is a major goal of modern scientific research. For example, by identifying genes and determining their sequences, scientists have been able to make large quantities of valuable human “gene products.” These include human insulin, interferon, Factor VIII, tumor necrosis factor, human growth hormone, tissue plasminogen activator, and numerous other compounds. Additionally, knowledge of gene sequences can provide the key to treatment or cure of genetic diseases (such as muscular dystrophy and cystic fibrosis).

In one aspect, the present invention is directed to each of the DNA sequences and molecules (and corresponding RNA sequences) identified in Table 2 and set forth in the Sequence Listing, and to fragments or portions of such sequences which contain at least 30 bases, and preferably at least 50 bases, and to those sequences which are at least 95% and preferably at least 97% identical thereto, and to DNA (RNA) sequences encoding the same polypeptide as the sequences of Table 2 as well as fragments and portions thereof. The sequences identified in Table 2 are hereinafter sometimes referred to as ESTs (Expressed Sequence Tags). Each such identified sequence is a sequenced portion of an overall cDNA sequence contained in a cDNA clone derived from human tissue. The three letter prefix of each EST correlates with the three letter code for the human tissues listed in Table 1, supra.

In accordance with a further aspect, the present invention is directed to a DNA sequence (as well as the corresponding RNA sequence) which is or contains a DNA sequence identical to one contained in and isolatable from ATCC Deposit No. 75916. The DNA sequence contained in the deposit is hybridizable under stringent conditions with a DNA sequence (EST) identified in Table 2 and set forth in the Sequence Listing. In addition, the present invention relates to fragments or portions of the isolated DNA sequences (and corresponding RNA sequences) containing at least 30 bases, preferably at least 40 bases and more preferably at least 50 bases, as well as sequences which are at least 97% identical thereto, as well as DNA (RNA) sequences encoding the same polypeptide.

As used herein, a first DNA (RNA) sequence is at least 95% and preferably at least 97% identical to another DNA (RNA) sequence if there is at least 95% and preferably at least a 95% or 97% identity, respectively, between the bases of the first sequence and the bases of the another sequence, when properly aligned with each other, for example when aligned by BLAST or FAST A.

In yet another aspect, the present invention is directed to an isolated DNA (RNA) sequence or molecule comprising at least the coding region of a human gene (or a DNA sequence encoding the same polypeptide as such coding region), in particular an expressed human gene, which human gene comprises a DNA sequence listed in Table 2 or one at least 95% and preferably at least 97% identical thereto, as well as fragments or portions of the coding region which encode a polypeptide having a similar function to the polypeptide encoded by the coding region. Thus, the isolated DNA (RNA) sequence can include only the coding region of the expressed gene (or fragment or portion thereof as hereinabove indicated) or can further include all or a portion of the non-coding DNA of the-expressed human gene.

In general, the sequences tabulated in Table 2 (or one at least 95% and preferably at least 97% identical thereto) are from the coding region of a human gene; however, it is to be understood that in some cases the sequence of Table 2 is in a non-coding region of a human gene. The isolated DNA of the present invention which is in the coding region or portion of such gene will not include the EST (or one at least 95% and preferably at least 97% identical thereto) if such EST is from the non-coding portion of the gene, even though such human gene is identified by use of such non-coding EST.

In yet another aspect, the present invention is directed to an isolated DNA sequence (RNA) containing at least the coding region of a human gene or a DNA (RNA) sequence encoding the same peptide as such coding region (in particular, an expressed human gene) which human gene (either in the coding or non-coding region and in general, in the coding region) contains a DNA sequence identical to a DNA sequence present in ATCC Deposit No. 75916, which DNA sequence in such ATCC Deposit is hybridizable under stringent conditions with a DNA sequence listed in Table 2. The invention further relates to fragments or portions of such coding region which encode a polypeptide having a similar function to the polypeptide encoded by the coding region.

The present invention further relates to polypeptides encoded by such hereinabove noted DNA (RNA) sequences, as well as the production and use of such polypeptides and fragments, derivatives and structural modifications thereof with the same function(s) and use(s) and to antibodies against such polypeptides.

The present invention also relates to vectors or plasmids which include such DNA (RNA) sequences, as well as the use of the DNA (RNA) sequences.

The material deposited as ATCC Deposit No. 75916 is a mixture of cDNA clones deposited as phages is derived from a variety of human tissue. The tissues from which the clones were derived are listed in Table 1, and the form of clone deposit from such tissue is also indicated in Table 1. The deposited material includes the cDNA clones which were partially sequenced and listed in Table 2. Thus, the DNA sequence of Table 2 is only a portion of the sequence included in the clone from which the sequence was derived. Thus, a clone which is isolatable from the ATCC Deposit by use of a sequence listed in Table 2 may include the entire coding region of a human gene or in other cases such clone may include a substantial portion of the coding region of a human gene. Although the sequence listing lists only a portion of the DNA sequence in a clone included in the ATCC Deposits, it is well within the ability of one skilled in the art to complete the sequence of the DNA included in a clone isolatable from the ATCC Deposits by use of a sequence (or portion thereof) listed in Table 2 by procedures hereinafter further described, and others apparent to those skilled in the art.

In addition, in the case where a clone isolatable from the ATCC Deposits by use of a DNA sequence (or portion thereof) listed in Table 2 does not include the full coding region of a human gene, it is well within the scope of those skilled in the art to obtain the full coding region by techniques described herein or others in the art relates to an isolated DNA.

The deposit which contains a clone which includes a DNA sequence of Table 2 can be determined from the library from which the clone and sequence was derived.

Because coding regions comprise such a small portion of the human genome, identification and mapping of transcribed regions and coding regions of chromosomes is of significant interest. There is a corresponding need for reagents for identifying and marking coding regions and transcribed regions of chromosomes. Furthermore, such human sequences are valuable for chromosome mapping, human identification, identification of tissue type and origin, forensic identification, and locating disease-associated genes (i.e., genes that are associated with an inherited human disease, whether through mutation, deletion, or faulty gene expression) on the chromosome.

The EST sequences disclosed herein are markers for and components of human genes actually transcribed in vivo. Techniques are disclosed for using these ESTs to obtain the full coding region of the corresponding gene. The use of ESTs, complete coding sequences, or fragments thereof for marking chromosomes, for mapping locations of expressed genes on chromosomes, for individual or forensic identification, for mapping locations of disease-associated genes, for identification of tissue type, and for preparation of antisense sequences, probes, and constructs is discussed in detail below. Unlike the random genomic DNA sequence tagged sites (STSs) (Olson et al., Science, 245:1434 (1989)), ESTs point directly to expressed genes.

Various aspects of the present invention thus include each of the individual ESTs, corresponding partial and complete cDNA, genomic DNA, mRNA, antisense strands, triple helix probes, PCR primers, coding regions, and constructs. Expression vectors and polypeptide expression products, are also within the scope of the present invention, along with antibodies, especially monoclonal antibodies, to such expression products.

The detailed description that follows provides not only the actual sequence of each new EST, but also explains

(i) how the ESTs were obtained,

(ii) how to obtain the corresponding complete coding region sequence and the corresponding genomic DNA sequence,

(iii) how to make DNA constructs from the ESTs and corresponding sequences,

(iv) how to use the ESTs and corresponding coding region sequences as therapeutics in gene therapy and resulting polypeptides and proteins as therapeutics,

(v) how to use those sequences as reagents in molecular biology and other fields, and

(vi) how to produce gene products from the ESTs and corresponding sequences and antibodies to those gene products.

Furthermore, numerous working examples are provided to demonstrate and exemplify various aspects of the invention.

As used herein and except as noted otherwise, the following terms have the following definitions.

As used herein, “enriched” means that the concentration of the material is at least about 2, 5, 10, 100, or 1000 times its natural concentration (for example) , advantageously 0.01%, by weight, preferably at least about 0.1% by weight. Enriched preparations of about 0.5%, 1%, 5%, 10%, and 20% by weight are also contemplated. The sequences, constructs, vectors, clones, and other materials comprising the present invention can advantageously be in enriched or isolated form. Further, removal of clones corresponding to ribosomal RNA and “housekeeping” genes and clones without human cDNA inserts results in a library that is “enriched” in the desired clones.

The term “isolated” means that the material is removed from its original environment (e.g., the natural environment if it is naturally occurring). For example, a naturally-occurring polynucleotide or DNA present in a living animal is not isolated, but the same polynucleotide or DNA, separated from some or all of the coexisting materials in the natural system, is isolated. Such DNA could be part of a vector and/or such polynucleotide could be part of a composition, and still be isolated in that such vector or polynucleotide is not part of its natural environment.

It is also advantageous that the sequences be in “purified” form. The term “purified” does not require absolute purity; rather, it is intended as a relative definition. Individual EST clones isolated from a cDNA library have been conventionally purified to electrophoretic homogeneity. The cDNA clones are obtained via manipulation of a partially purified naturally occurring substance (messenger RNA). By conversion of mRNA into a cDNA library, pure individual cDNA clones can be isolated from the synthetic library by clonal selection. Thus, creating a cDNA library from RNA and subsequently isolating individual clones from that library results in an approximately 10⁶ fold purification of the native message. Purification of starting material or natural material to at least one order of magnitude, preferably two or three orders, and more preferably four or five orders of magnitude is expressly contemplated. Furthermore, claimed polynucleotide which has a purity of preferably 0.001%, or at least 0.01% or 0.1%; and even desirably 1% by weight or greater is expressly contemplated.

The term “coding region” refers to that portion of a human gene which either naturally or normally codes for the expression product of that gene in its natural genomic environment, i.e., the region coding in vivo for native expression product of the gene. The coding region can be from a normal, mutated or changed gene.

The term “gene” or “cistron” means the segment of DNA involved in producing a polypeptide chain; it includes regions preceding and following the coding region (leader and trailer) as well as intervening sequences (introns) between individual coding segments (exons).

The term “expression product” means that polypeptide or protein that is the natural transcription product of the gene and any nucleic acid sequence coding equivalents based on degeneracy of the code coding for the same amino acid(s).

The term “fragment” when referring to a coding sequence means a portion of DNA comprising less than the complete human coding region whose expression product retains essentially the same biological function or activity as the expression product of the complete coding region.

The term “primer” means a short nucleic acid sequence that is paired with one strand of DNA and provides a free 3′ OH end at which a DNA polymerase starts synthesis of a deoxyribonucleotide chain.

The term “promoter” means a region of DNA involved in binding of RNA polymerase to initiate transcription.

The term “open reading frame (ORF)” means a series of triplets coding for amino acids without any termination codons and is a sequence (potentially) translatable into protein.

The term “oncogene” means genes whose products have the ability to transform eukaryotic cells so that they grow in a manner analogous to tumor cells. Oncogenes carried by retroviruses have names of the form v-onc. Proto-oncogenes are the normal counterparts in the eukaryotic genome to the oncogenes carried by some retroviruses. They are given names of the form c-onc.

The term “exon” means any segment of an interrupted gene that is represented in the mature RNA product.

As used herein reference to a DNA sequence includes both single stranded and double stranded DNA. Thus, the specific sequence, unless the context appears otherwise refers to the single strand DNA of such sequence, the duplex of such sequence with its complement (double stranded DNA) and the complement of such sequence.

I. ESTs are Obtained from cDNA Libraries

The EST sequences of the present invention have been isolated from custom made and commercially available cDNA libraries using a rapid screening and sequencing technique. In general, the method comprises applying automated DNA sequencing technology to screen clones, advantageously randomly selected clones, from a cDNA library. Preferably, the library is initially “enriched” by removal of ribosomal sequences and other common sequences prior to clone selection. According to the disclosed method, ESTs are generated from partial DNA sequencing of the selected clones. The ESTs of the present invention were generated using low redundancy of sequencing, typically a single sequencing reaction. While single sequencing reactions may have an accuracy as low as 97%, this nevertheless provides sufficient fidelity for identification of the sequence and design of PCR primers, as well as for full length sequence because of the exceptional amount of laboratory work and resultant chemical/biological disclosure reported herein, including that done by automatically cycle sequencing.

The automated sequencing reported here was performed on catalyst robots (Applied Biosystems, Inc., Foster City, Calif.) and 373 Automated DNA Sequencers (Applied Biosystems, Inc.). The Catalyst robot is a sophisticated pipetting and temperature controlled robot that has been developed specifically for DNA sequencing reactions. The Catalyst combines pre-aliquoted templates and reaction mixtures consisting of deoxy- and dideoxynucleotides, the Taq thermostable DNA polymerase, fluorescently-labelled sequencing primers, and reaction buffer. Reaction mixtures and templates are combined in the wells of an aluminum 96-well thermocycling plate. Thirty consecutive cycles of linear amplification (e.g. one primer synthesis) steps are performed including denaturation, annealing of primer and template, and extension of DNA synthesis. A heated lid on the thermocycling plate prevents evaporation without the need for an oil overlay. The Applied Biosystems, Inc. (ABI) system currently used for EST sequencing involves use of four dye-labelled sequencing primers, one for each of the four terminator nucleotides. Each dye-primer is labelled with a different fluorescent dye, permitting the four individual reactions to be combined into one lane of the 373 DNA Sequencer for electrophoresis, detection, and base-calling. ABI supplies pre-mixed reaction mixes (PRIZM Ready Reaction Kit) containing all the necessary non-template reagents for sequencing. These reaction mixtures are stable for at least a year at −20° C.

Between 24 and 36 samples are loaded onto each 373 Sequencer each day. Electrophoresis is run overnight, and data are collected for twelve hours. Following electrophoresis and fluorescence detection, the 373 sequencer performs automatic lane tracking and base-calling. The lane-tracking is confirmed visually and data are archived to 8 mm tape daily. Each sequence chromatogram (or fluorescence lane trace) is inspected visually and assessed for quality. Leading vector polylinker sequence and trailing sequence of low quality are removed and the sequence itself is loaded via software into the EST database (estdb) which is described more fully below. Average edited lengths of sequences from the 373 sequencers are about 400 bp and depend most on the quality of the template used for the sequencing reaction. Thus depending on the length of the polylinker, ESTs of up to 370 bp are generated by single sequencing runs (assuming 30 bp polylinker is removed).

ESTs comprise DNA sequences corresponding to a portion of nuclear encoded messenger RNA. An EST is of sufficient length to permit: (1) amplification of the specific sequence from a cDNA library, e.g., by polymerase chain reaction (PCR); (2) use of a synthetic polynucleotide corresponding to a partial or complete sequence of the EST as a hybridization probe of a cDNA library, generally having about 30-50 base pairs; or (3) unique designation of the pure cDNA clone from which the EST was derived (the EST clone) for use as a hybridization probe of a cDNA library. The length of a partial EST according to the present invention can be, for example, approximately 30, 40, 50, 75, 90, 100, or 150 bases. Preferably, EST-derived primer pairs and sequences amplify or detectably hybridize to a sequence from a genomic library.

It has been found that sufficient information is contained in the 150-400 base ESTs from one sequencing run to effect preliminary identification and exact chromosome mapping. Accordingly, the ESTs disclosed herein are generally at least 150 base pairs in length. The length of an EST is determined by the quality of sequencing data and the length of the cloned cDNA. Raw data from the automated sequencers are edited to remove low quality sequence at the end of the sequencing run. High quality sequences (usually a result of sequencing templates without excessive salt contamination) generally give about 400 bp of reliable sequence data; other sequences give fewer bases of reliable data. A 150 bp EST is long enough to be translated into a 50 amino acid peptide sequence. This length is sufficient to observe similarities when they exist in a database search. Furthermore, 150 bp is long enough to design PCR primers from each end of the sequence to amplify the complete EST. Sequences shorter than 150 bp are difficult to purify and use following PCR amplification. Furthermore, a 150 bp polynucleotide is likely to give a very strong signal with low background in a screen of a genomic library.

Finally, it is highly unlikely that a sequence of the same 150 bp exists in any genes in the genome besides the one tagged by the EST. Some closely related gene family members have very similar nucleotide sequences, but no examples of pairs of human genes with long segments of identical sequence have been reported to date.

ESTs that match perfectly to several different genes can be detected by hybridizing to chromosomes: if many chromosomal loci are observed, the sequence (or a close variant) is in more than one gene. This problem can be circumvented by using the 3′-untranslated part of the cDNA alone as a probe for the chromosomal location or for the full-length cDNA or gene. The 3′-untranslated region is more likely to be unique within gene families, since there is no evolutionary pressure to conserve a coding function of this region of the mRNA.

As demonstrated in the Examples that follow, ESTs can be used to map the expressed sequence to a particular chromosome. In addition, ESTs can be expanded to provide the full coding regions, as detailed below. Previously unknown genes are identified in this manner.

While a variety of cDNA libraries can be used to obtain ESTs, the cDNA libraries listed below are exemplified and represent a preferred embodiment. Suitable cDNA libraries can be freshly prepared or obtained commercially. The cDNA libraries from the desired tissue are preferably preprocessed by conventional techniques to reduce repeated sequencing of high and intermediate abundance clones and to maximize the chances of finding rare messages from specific cell populations. Preferably, preprocessing includes the use of defined composition prescreening probes, e.g., cDNA corresponding to mitochondria, abundant sequences, ribosomes, actins, myelin basic polypeptides, or any other known high abundance peptide; these prescreening probes used for preprocessing are generally derived from known ESTs. Other useful preprocessing techniques include subtraction, which preferentially reduces the population of certain sequences in the library (e.g., see A. Swaroop et al., Nucl. Acids Res., 19: 1954 (1991)), and normalization, which results in all sequences being represented in approximately equal proportions in the library (Patanjali et al, Proc. Natl. Acad. Sci. USA, 88:1943 (1991)).

The cDNA libraries used in the present method ideally use directional cloning methods so that either the 5′ end of the cDNA (likely to contain coding sequence) or the 3′ end (likely to be a non-coding sequence) can be selectively obtained.

Libraries of cDNA can also be generated from recombinant expression of genomic DNA. After they are amplified, ESTs can be obtained and sequenced, e.g., as illustrated in Example 9.

The sequences of the present invention include each of the specific sequences set forth in the Sequence Listing and designated SEQ ID NO:1-SEQ ID NO: 7483. In one aspect of this embodiment, the invention relates to those sequences of SEQ ID NOS:1-7483 that are part of the cDNA coding sequences for polypeptides where the polypeptide encoded by the EST has less than 95% identity and preferably also less than 95% similarity to a polypeptide sequence encoded by a known corresponding DNA sequence (see ESTs in Table 2) and more preferably less than 90% or 85% identity. In another aspect, the invention relates to those sequences of SEQ ID NOS:1-7483 that have less than 95% identity with known DNA sequences. As used herein, the term “similarity” with respect to amino acid sequences means that an amino acid sequence and conserved amino acid substituents thereof are compared to another amino acid sequence. Thus, an amino acid sequence and substituted conservative amino acid are compared to another amino acid sequence to determine “similarity.”

II. Complete Coding Region DNA Sequences Recovered Using ESTs

The ESTs of the present invention generally represent relatively small coding regions or untranslated regions of human genes. Although these EST sequences do not generally code for a complete gene product, they are highly specific markers for the corresponding complete coding regions. The ESTs are of sufficient length that they will hybridize, under stringent conditions, only with DNA for that gene to which they correspond Suitably stringent conditions comprise conditions, for example, where at least 95%, preferably at least 97% or 98% identity (base pairing), is required for hybridization. This property permits use of the EST to isolate the entire coding region and even the entire sequence. Therefore, only routine laboratory work is necessary to parlay the unique EST sequence into the corresponding unique complete gene sequence.

Thus, each of the ESTs of the present invention “corresponds” to or is a part of a particular unique human gene. Knowledge of the EST sequence permits isolation and sequencing of the complete coding sequence of the corresponding gene. The complete coding sequence is present in a full-length cDNA clone as well as in the gene carried on genomic clones. Therefore, each EST also “corresponds” to or is a part of a complete genomic gene sequence, and may or may not be DNA which is included in a polypeptide coding region of the gene.

The first step in determining where an EST is located in the cDNA is to analyze the EST for the presence of coding sequence, e.g., as described in Example 12. The CRM program predicts the extent and orientation of the coding region of a sequence. Based on this information, one can infer the presence of start or stop codons within a sequence and whether the sequence is completely coding or completely noncoding. If start or stop codons are present, then the EST can cover both part of the 5′-untranslated or 3′-untranslated part of the mRNA (respectively) as well as part of the coding sequence. If no coding sequence is present, it is likely that the EST is derived from the 3′-untranslated sequence due to its longer length and the fact that most cDNA library construction methods are biased toward the 3′ end of the mRNA.

An EST is a specific tag for a messenger RNA molecule. The complete sequence of that messenger RNA, in the form of cDNA, can be determined using the EST as a probe to identify a cDNA clone corresponding to a full-length transcript, followed by sequencing of that clone. The EST or the fulllength cDNA clone can also be used as a probe to identify a genomic clone or clones that contain the complete gene including regulatory and promoter regions, exons, and introns.

ESTs are used as probes to identify the cDNA clones from which an EST was derived. ESTs, or portions thereof, can be nick-translated or end-labelled with ³²P using polynucleotide kinase using labelling methods known to those with skill in the art (Basic Methods in Molecular Biology, L. G. Davis, M. D. Dibner, and J. F. Battey, ed., Elsevier Press, NY, 1986). A lambda library can be directly screened with the labelled ESTs of interest or the library can be converted en masse to pBluescript (Stratagene Cloning Systems, 11099 N. Torrey Pines Road, La Jolla, Calif. 92037) to facilitate bacterial colony screening. Regarding pBluescript, see Sambrook et al., Molecular Cloning-A Laboratory Manual, Cold Spring Harbor Laboratory Press (1989), pg. 1.20. Both methods are well known in the art. Briefly, filters with bacterial colonies containing the library in pBluescript or bacterial lawns containing lambda plaques are denatured and the DNA is fixed to the filters. The filters are hybridized with the labelled probe using hybridization conditions described by Davis et al., supra. The ESTs, cloned into lambda or pBluescript, can be used as positive controls to assess background binding and to adjust the hybridization and washing stringencies necessary for accurate clone identification. The resulting autoradiograms are compared to duplicate plates of colonies or plaques; each exposed spot corresponds to a positive colony or plaque. The colonies or plaques are selected, expanded and the DNA is isolated from the colonies for further analysis and sequencing.

The ESTs can additionally be used to screen Northern blots of mRNA obtained from various tissues or cell cultures, including the tissue of origin of the EST clone. Northern analysis will most often produce one to several positive bands. The bands can be selected for further study based on the predicted size of the mRNA.

Positive cDNA clones in phage lambda are analyzed to determine the amount of additional sequence they contain using PCR with one primer from the EST and the other primer from the vector. Clones with a larger vector-insert PCR product than the original EST clone are analyzed by restriction digestion and DNA sequencing to determine whether they contain an insert of the same size or similar as the mRNA size on a Northern blot.

Once one or more overlapping cDNA clones are identified, the complete sequence of the clones can be determined. The preferred method is to use exonuclease III digestion (McCombie, W. R, Kirkness, E., Fleming, J. T., Kerlavage, A. R., Iovannisci, D. M., and Martin-Gallardo, R., Methods, 3:33-40, 1991). A series of deletion clones is generated, each of which is sequenced. The resulting overlapping sequences are assembled into a single contiguous sequence of high redundancy (usually three to five overlapping sequences at each nucleotide position), resulting in a highly accurate final sequence.

A similar screening and clone selection approach can be applied to obtaining cosmid or lambda clones from a genomic DNA library that contains the complete gene from which the EST was derived (Kirkness, E. F., Kusiak, J. W., Menninger, J., Gocayne, J. D., Ward, D. C., and Venter, J. C., Genomics 10: 985-995 (1991). Although the process is much more laborious, these genomic clones can be sequenced in their entirety also. A shotgun approach is preferred to sequencing clones with inserts longer than 10 kb (genomic cosmid and lambda clones) . In shotgun sequencing, the clone is randomly broken into many small pieces, each of which is partially sequenced. The sequence fragments are then aligned to produce the final contiguous sequence with high redundancy. An intermediate approach is to sequence just the promoter region and the intron-exon boundaries and to estimate the size of the introns by restriction endonuclease digestion (ibid.).

Using the sequence information provided herein, the polynucleotides of the present invention can be derived from natural sources or synthesized using known methods. The sequences falling within the scope of the present invention are not limited to the specific sequences described, but include human allelic and species variations thereof and portions thereof of at least 15-18 bases, preferably at least 25, 40, or 50 bases, and more preferably at least 75, 90, 100, 125, or 150 bases. (Sequences of at least 15-18 bases can be used, for example, as PCR primers or as DNA probes.) In addition, the invention includes the entire coding sequence associated with the specific polynucleotide sequence of bases described in the Sequence Listing, as well as portions of the entire coding sequence of at least 15-18 bases, preferably at least 25, 40, or 50 bases, and more preferably at least 75, 90, 100, 125, or 150 bases, and allelic and species variations thereof. Allelic variations can be routinely determined by comparison of one sequence with a sequence from another individual of the same species. Furthermore, to accommodate codon variability, the invention includes sequences coding for the same amino acid sequences as do the specific sequences disclosed herein. In other words, in a coding region, substitution of one codon for another which encodes the same amino acid is expressly contemplated. (Coding regions can be determined through routine sequence analysis.)

Any specific sequence disclosed herein can be readily screened for errors by resequencing each EST in both directions (i.e., sequence both strands of cDNA). Alternatively, error screening can be performed by sequencing corresponding polynucleotide of human origin isolated by using part or all of the EST in question as a probe or primer.

In a cDNA library there are many species of mRNA represented. Each cDNA clone can be interesting in its own right, but must be isolated from the library before further experimentation can be completed. In order to sequence any specific cDNA, it must be removed and separated (i.e. isolated and purified) from all the other sequences. This can be accomplished by many techniques known to those of skill in the art. These procedures normally involve identification of a bacterial colony containing the cDNA of interest and further amplification of that bacteria. Once a cDNA is separated from the mixed clone library, it can be used as a template for further procedures such as nucleotide sequencing.

Although claims to large numbers of ESTs and corresponding sequences are presented herein, the invention is not limited to these particular groupings of sequences. Thus, individual sequences are considered as applicants' discoveries or inventions, as are subgroupings of sequences.

III. DNA Constructs

The present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including for example, a promoter, operably linked to the sequence. Large numbers of suitable vectors and promoters are known to those of skill in the art, and are commercially available. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, pXT1, pSG (Stratagene) pSVK3, pBPV, pMSG, pSVL (Pharmacia).

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described construct. The host cell can be a higher eukaryotic cell, such as a mammalian cell, or a lower eukaryotic cell, such as a yeast cell, or the host cell can be a procaryotic cell, such as a bacterial cell. Introduction of the construct into the host cell can be effected by calcium phosphate transfection, DEAE, dextran mediated transfection, or electroporation (Davis, L., Dibner, M., Battey, I., Basic Methods in Molecular Biology, 1986))

The constructs in host cells can be used in a conventional manner to produce the gene product coded by the recombinant sequence. Alternatively, the encoded polypeptide can be synthetically produced by conventional peptide synthesizers.

IV. ESTs and Corresponding Sequences as Reagents

Each of the cDNA sequences identified herein (and the corresponding complete gene sequences) can be used in numerous ways as polynucleotide reagents. The sequences can be used as diagnostic probes for the presence of a specific mRNA in a particular cell type. In addition, these sequences can be used as diagnostic probes suitable for use in genetic linkage analysis (polymorphisms). Further, the sequences can be used as probes for locating gene regions associated with genetic disease, as explained in more detail below.

The ESTs and complete gene sequences of the present invention are also valuable for chromosome identification. Each sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of ESTs and cDNAs to chromosomes according to the present invention is an important first step in correlating those sequences with genes associated with disease.

Briefly, sequences can be mapped to chromosomes by preparing PCR primers (preferably 15-25 bp) from the ESTs. Computer analysis of the ESTs is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the EST will yield an amplified fragment.

PCR mapping of somatic cell hybrids is a rapid procedure for assigning a particular EST to a particular chromosome. Three or more clones can be assigned per day using a single thermal cycler. Using the present invention with the same oligonucleotide primers, sublocalization can be achieved with panels of fragments from specific chromosomes or pools of large genomic clones in an analogous manner. Other mapping strategies that can similarly be used to map an EST to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 500 or 600 bases; however, clones larger than 2,000 bp have a higher likelihood of binding to a unique chromosomal location with sufficient signal intensity for simple detection. FISH requires use of the clone from which the EST was derived, and the longer the better. For example, 2,000 bp is good, 4,000 is better, and more than 4,000 is probably not necessary to get good results a reasonable percentage of the time. For a review of this technique, see Verma et al., Human Chromosomes: a Manual of Basic Techniques. Pergamon Press, New York (1988).

Reagents for chromosome mapping can be used individually (to mark a single chromosome or a single site on that chromosome) or as panels of reagents (for marking multiple sites and/or multiple chromosomes). Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man (available on line through Johns Hopkins University Welch Medical Library).) The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb.)

Comparison of affected and unaffected individuals generally involves first looking for structural alterations in the chromosomes, such as deletions or translocations that are visible from chromosome spreads or detectable using PCR based on that cDNA sequence. Ultimately, complete sequencing of genes from several individuals is required to, confirm the presence of a mutation and to distinguish mutations from polymorphisms.

In addition to the foregoing, the sequences of the invention, as broadly described, can be used to control gene expression through triple helix formation or antisense DNA or RNA, both of which methods are based on binding of a polynucleotide sequence to DNA or RNA. Polynucleotides suitable for use in these methods are usually 20 to 40 bases in length and are designed to be complementary to a region of the gene involved in transcription (triple helix—see Lee et al, Nucl. Acids Res., 6:3073 (1979); Cooney et al, Science, 241:456 (1988); and Dervan et al, Science, 251: 1360 (1991)) or to the mRNA itself (antisense—Okano, J. Neurochem., 56:560 (1991); Oligodeoxynucleotides as Antisense Inhibitors of Gene Expression, CRC Press, Boca Raton, Fla. (1988)). Triple helix—formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide.

The present invention is also a useful tool in gene therapy, which requires isolation of the disease-associated gene in question as a prerequisite to the insertion of a normal gene into an organism to correct a genetic defect. The high specificity of the cDNA probes according to this invention have promise of targeting such gene locations in a highly accurate manner.

The sequences of the present invention, as broadly defined, are also useful for identification of individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The sequences of the present invention are useful as additional DNA markers for RFLP.

However, RFLP is a pattern based technique, which does not require the DNA sequence of the individual to be sequenced. The sequences of the present invention can be used to provide an alternative technique that determines the actual base-by-base DNA sequence of selected portions of an individual's genome. These sequences can be used to prepare PCR primers for amplifying and isolating such selected DNA. One can, for example, take an EST of the invention and prepare two PCR primers from the 5′ and 3′ ends of the EST. These are used to amplify an individual's DNA, corresponding to the EST. The amplified DNA is sequenced.

Panels of corresponding DNA sequences from individuals, made this way, can provide unique individual identifications, as each individual will have a unique set of such DNA sequences, due to allelic differences. The sequences of the present invention can be used to particular advantage to obtain such identification sequences from individuals and from tissue, as further described in the Examples. The EST sequences from Example 1 and the complete sequences from Examples 3 and 9 uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the ESTs or complete coding sequences comprising a part of the present invention can, to some degree, be used as a standard against which DNA from an individual can be compared for identification purposes. Because greater numbers of polymorphisms occur in the noncoding regions, fewer sequences are necessary to differentiate individuals.

If a panel of reagents from ESTs or complete sequences of this invention is used to generate a unique ID database for an individual, those same reagents can later be used to identify tissue from that individual. Positive identification of that individual, living or dead can be made from extremely small tissue samples.

Another use for DNA-based identification techniques is in forensic biology. PCR technology can be used to amplify DNA sequences taken from very small biological samples such as tissues, e.g., hair or skin, or body fluids, e.g., blood, saliva, semen, etc. In one prior art technique, gene sequences are amplified at specific loci known to contain a large number of allelic variations, for example the DQα class II HLA gene (Erlich, H., PCR Technology, Freeman and Co. (1992)). Once this specific area of the genome is amplified, it is digested with one or more restriction enzymes to yield an identifying set of bands on a Southern blot probed with DNA corresponding to the DQα class II HLA gene.

The sequences of the present invention can be used to provide polynucleotide reagents specifically targeted to additional loci in the human genome, and can enhance the reliability of DNA-based forensic identifications. Those sequences targeted to noncoding regions are particularly appropriate. As mentioned above, actual base sequence information can be used for identification as an accurate alternative to patterns formed by restriction enzyme generated fragments. Reagents for obtaining such sequence information are within the scope of the present invention. Such reagents can comprise complete genes, ESTs or corresponding coding regions, or fragments of either of at least 15 bp, preferably at least 18 bp.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from the ESTs or complete sequences of the present invention. Panels of such reagents can identify tissue by species and/or by organ type. In a similar fashion, these reagents can be used to screen tissue cultures for contamination.

V. Production of Polypeptide Corresponding to ESTs

Once the coding sequence is known, or the gene is cloned which encodes the polypeptide, conventional techniques in molecular biology can be used to obtain the polypeptide.

At the simplest level, the amino acid sequence can be synthesized using commercially available peptide synthesizers. This is particularly useful in producing small peptides and fragments of larger polypeptides. (Fragments are useful, for example, in generating antibodies against the native polypeptide.)

Alternatively, the DNA encoding the desired polypeptide can be inserted into a host organism and expressed. The organism can be a bacterium, yeast, cell line, or multicellular plant or animal. The literature is replete with examples of suitable host organisms and expression techniques. For example, polynucleotide (DNA or mRNA) can be injected directly into muscle tissue of mammals, where it is expressed. This methodology can be used to deliver the polypeptide to the animal, or to generate an immune response against a foreign polypeptide. Wolff, et al., Science, 247:1465 (1990); Felgner, et al., Nature, 349:351 (1991). Alternatively, the coding sequence, together with appropriate regulatory regions (i.e., a construct), can be inserted into a vector, which is then used to transfect a cell. The cell (which may or may not be part of a larger organism) then expresses the polypeptide. (See Example 23.) Such techniques are discussed in more detail below.

VI. Recombinant Production Techniques and Purification

“Substantially equivalent,” can refer both to nucleic acid and amino acid sequences, for example a mutant sequence, that varies from a reference sequence by one or more substitutions, deletions, or additions, the net effect of which does not result in an adverse functional dissimilarity between reference and subject sequences. For purposes of the present invention, sequences having equivalent biological activity, and equivalent expression characteristics are considered substantially equivalent. For purposes of determining equivalence, truncation of the mature sequence should be disregarded.

“Recombinant,” as used herein, means that a protein is derived from recombinant (e.g., microbial or mammalian) expression systems. “Microbial” refers to recombinant proteins made in bacterial or fungal (e.g., yeast) expression systems. As a product, “recombinant microbial” defines a protein essentially free of native endogenous substances and unaccompanied by associated native glycosylation. Protein expressed in most bacterial cultures, e.g., E. coli, will be free of glycosylation modifications; protein expressed in yeast will have a glycosylation pattern different from that expressed in mammalian cells.

“DNA segment” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, using a cloning vector. Such segments are provided in the form of an open reading frame uninterrupted by internal nontranslated sequences, or introns, which are typically present in eukaryotic genes. Sequences of non-translated DNA may be present downstream from the open reading frame, where the same do not interfere with manipulation or expression of the coding regions.

“Nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

“Recombinant expression vehicle or vector” refers to a plasmid or phage or virus or vector, for expressing a polypeptide from a DNA (RNA) sequence. The expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

“Recombinant expression system” means host cells which have stably integrated a recombinant transcriptional unit into chromosomal DNA or carry the recombinant transcriptional unit extra chromosomally. The cells can be prokaryotic or eukaryotic. Recombinant expression systems as defined herein will express heterologous protein upon induction of the regulatory elements linked to the DNA segment or synthetic gene to be expressed.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryatic and eukaryotic hosts are described by Sambrook et al., Molecular Cloning: A Laboratory Manual, Second Edition, (Cold Spring Harbor, N.Y., 1989), the disclosure of which is hereby incorporated by reference.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may, also be employed as a matter of choice.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter is derepressed by appropriate means (e.g., temperature shift or chemical induction) and cells are cultured for an additional period. Cells are typically harvested by centrifugation, disrupted by physical or chemical means, and the resulting crude extract retained for further purification.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements.

Recombinant protein produced in bacterial culture is usually isolated by initial extraction from cell pellets, followed by one or more salting-out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents.

VII. Antibody Production and Use

The protein, its fragments or other derivatives, or analogs thereof, or cells expressing them can be used as an immunogen to produce antibodies thereto. These antibodies can be, for example, polyclonal, monoclonal, chimeric, single chain, Fab fragments, or the product of an Fab expression library. Various procedures known in the art may be used for the production of polyclonal antibodies.

Antibodies generated against the polypeptide corresponding to a sequence of the present invention can be obtained by direct injection of the polypeptide into an animal or by administering the polypeptide to an animal, preferably a nonhuman. The antibody so obtained will then bind the polypeptide itself. In this manner, even a sequence encoding only a fragment of the polypeptide can be used to generate antibodies binding the whole native polypeptide. Such antibodies can then be used to isolate the polypeptide from tissue expressing that polypeptide. Moreover, a panel of such antibodies, specific to a large number of polypeptides, can be used to identify and differentiate such tissue.

For preparation of monoclonal antibodies, any technique which provides antibodies produced by continuous cell line cultures can be used. Examples include the hybridoma technique (Kohler and Milstein, 1975, Nature, 256:495-497) , the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention.

The antibodies can be used in methods relating to the localization and activity of the protein sequences of the invention, e.g., for imaging these proteins, measuring levels thereof in appropriate physiological samples and the like.

As hereinabove indicated, the sequences of Table 2 are a portion of an expressed human gene and a DNA sequence including at least the coding region from such human gene can be used to produce a polypeptide expression product.

The present invention also provides pharmaceutical compositions. Such compositions comprise a therapeutically effective amount of the protein, and a pharmaceutically acceptable carrier or excipient. Such a carrier includes but is not limited to saline, buffered saline, dextrose, water, glycerol, ethanol, and combinations thereof. The formulation should suit the mode of administration.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

Certain aspects of the present invention are described in greater detail in the non-limiting Examples that follow.

### EXAMPLE 1

### cDNA Sequences Determined by Random Clone Selection

Preparation of cDNA Libraries

Tissues and cells used for preparation of RNA were obtained from various sources including the National Disease Research Interchange, Cooperative Human tissue Network, and the American Red Cross. In order to ensure the integrity of the RNA tissues, only samples that were snap frozen in liquid nitrogen were obtained and fresh samples of blood products were used. Total cellular RNA was prepared fro tissues by the guanidinium-phenol method as previously described (P. Chomczynski and N. Sacchi, Anal. Biochem., 162: 156-159 (1987)) using RNAzol (Cinna-Biotecx) and an additional ethanol precipitation of the RNA was included. Poly A mRNA was isolated from the total RNA using oligo dT-coated latex beads (Qiagen). Two rounds of poly A selection were performed to ensure better separation from non-polyadenylated material when sufficient quantities of total RNA were available.

The mRNA selected on the oligo dT was used for the synthesis of cDNA by a modification of the method of Gubler and Hoffman (Gubler, U. and B. J. Hoffman, 1983, Gene, 25:263). The first strand synthesis was performed using either Moloney murine reverse transcriptase (Stratagene) or Superscript II (RNase H minus Moloney murine reverse transcriptase, Gibco-BRL). First strand synthesis was primed using a primer/linker containing an Xho I restriction site. The nucleotide mix used in the synthesis contains methylated dCrP to prevent restriction within the cDNA sequence. For second-strand synthesis E. coli polymerase Klenow fragment was used and [³²P] -dATP was incorporated as a tracer-of nucleotide incorporation.

Following 2nd strand synthesis the cDNA was made blunt ended using either T4 DNA polymerase or Klenow fragment. Eco RI adapters were added to the cDNA and the cDNA was restricted with Xho I. The cDNA was size fractionated over a Sephacryl S-500 column (Pharmacia) to remove excess linkers and cDNAs under approximately 500 base pairs.

The cDNA was cloned unidirectionally into the Eco RI-Xho I sites of either pBluescript II phagemid or lambda Unizap XR (Stratagene). In the case of cloning into pBluescript II, the plasmids were electroporated into E. coli SURE competent cells (Stratagene). When the cDNA was cloned into Uni-Zap XR it was packaged using the Gigipack II packaging extract (Stratagene). The packaged phage were used to infect Sure cells and amplified. The pBluescript phagemid containing the cDNA inserts are excised from the lambda Zap phage using the helper phage ExAssist (Stratagene). The rescued phagemid is plated on SOLR E. coli cells (Statagene).

Preparation of Sequencing Templates

Template DNA for sequencing was prepared by 1) a boiling method or 2) PCR amplification.

The boiling method was a modification of the method of Holmes and Quigley (Holmes, D. S. and M. Quigley, 1981, Anal. Biochem., 114:193). Colonies from either cDNA cloned into Bluescript II or rescued Bluescript phagemid were grown in an enriched bacterial media overnight. 400 μl of cells were centrifuged and resuspended in STET (0.1M NaCl, 10 mM TRIS Ph 8.0, 1.0 nM EDTA and 5% Triton X-100) including lysozyme (80 μg/ml) and RNase A (4 μg/ml). Cells were boiled for 40 seconds and centrifuged for 10 minutes. The supernatant was removed and the DNA was precipitated with PEG/NaCl and washed with 70% ethanol (2×). Templates were resuspended in water at approximately 250 ng/μl.

Preparation of templates by PCR was a modification of the method of Rosenthal, et al (Rosenthal, et al., Nucleic Acids Res., 1993, 21:173-174). Colonies containing cDNA cloned into pBluescript II or rescued pBluescript phagemid were grown overnight in LB containing ampicillin in a 96 well tissue culture plate. Two μl of the cultures were used as template in PCR reaction (Saiki, R K, et al., Science, 239:487-493, 1988; and Saiki, R K, et al., Science, 230:1350-1354, 1985) using a tricine buffer system (Ponce and Micol, Nucleic Acids Res., 1992, 20:1992.) and 200 uM dNTPs. The primer set chosen for amplification of the templates was outside of primer sites chosen for sequencing of the templates. The primers used were 5′-ATGCTTCCGGCTCGTATG-3′ which is 5′ of the M13 reverse sequence in pBluescript and 5′-GGGTTTTCCCAGTCACGAC-3′, which is 3 prime of the M13 forward primer in pBluescript. Any primers which correspond to the sequence flanking the M13 forward and reverse sequences could be used. Perkin-Elmer 9600 thermocyclers were used for amplification of the templates with the following cycler conditions: 5 min at 94° C. (1 cycle); (20 sec at 94° C.); 20 sec at 55° C. (1 min at 72° C.) (30 cycles) ; 7 min at 72° C. (1 cycle). Following amplification the PCR templates were precipitated using PEG/NaCl and washed three times with 70% ethanol. The templates were resuspended in water.

The several human cDNA libraries giving assigned Library IDs (Lib. ID) and the tissue used as sources of clones for sequencing are set forth in Table 1.

Results

A directional library would be expected to contain a bias toward coding sequence at the 5′ end of the insert relative to the 3′ end. Two measures of coding content, peptide database matches (obtained by searching a comprehensive database with the “basic local alignment search tool” BLAST (Altschul, et al., J. Mol. Biol., 215:403-410, 1990), and the GRAIL coding-region prediction program (Uberbacher, et al., Proc, Nat'l Acad. Sci. USA, 88:11261-11265, 1991) were used to estimate the coding percentage of 5′ and 3′ end sequences, as explained in Example 2.

TABLE 1

| | | |
|---|---|---|
| H0009 | HFCA HFCB HFCC HFCD HFCE HFCF | Human Fetal |
| | | Brain (II) |
| H0012 | HFKC HFKD HFKE | Human Fetal Kidney |
| H0014 | HGBA HGBD HGBE HGBF HGBG | Human Gall Bladder |
| H0024 | HLHA HLHB HLHC HLHD HLHH HLHQ | Human Fetal Lung III |
| H0032 | HPRA HPRB HPRC HPRD | Human Prostate |
| H0038 | HTEA HTEB HTEC HTED HTEE HTEF | Human Testes |
| H0039 | HTPA HTPB HTPC | Human Pancreas Tumor |
| H0040 | HTTA HTTB HTTC HTTD | Human Tumor Testes |
| H0042 | HAPA HAPB HAPC HAPM | Human Adult Pulmonary |
| H0046 | HETA HETB HETC HETD HETE HETF | Human Edometrial Tumor |
| H0050 | HHFB HHFC HHFD HHFE HHFF | Human Fetal Heart |
| H0051 | HHPB HHPC HHPD HHPE HHPF HHPG | Human Hippocampus |
| H0052 | HCEB HCEC HCED HCEE HCEF HCEG | Human Cerebellum |
| H0058 | HUVB HUVC HUVD HUVE | Human Umbilical Vein, |
| | | Endo. remake |
| H0059 | HUKA HUKB HUKC HUKD | Human uterine cancer |
| H0068 | HSTA HSTB HSTC | Human Skin Tumor |
| H0069 | HTAA HTAB HTAC | Human Activated T-Cells |
| H0075 | HTBA HTBB | Human Activated T-Cells |
| | | (II) |
| H0081 | HFEA | Human Fetal Epithelium |
| | | (Skin) |
| H0085 | HCNA HCNB | Human Colon |
| H0090 | HLTA HLTB HLTC | Human T-Cell Lymphoma |
| H0123 | HFTA HFTB | Human Fetal Dura Mater |
| H0134 | HRGA HRGB HRGC | Raji cells, |
| | | cyclohexamide treated |
| H0135 | HSSA HSSB HSSC HSSD HSSM HSSN | Human Synovial Sarcoma |
| H0141 | HT4A HT4C HT4D | Activated T-Cells, 12 |
| | | Hrs. |
| H0144 | HE9A HE9B HE9C HE9D HE9E HE9F | Nine week old Early |
| | | Stage Human |
| H0149 | HE7T | Seven week old Early |
| | | Stage Human, subracted |
| H0150 | HEPA HEPB HEPC | Human Epididymus |
| H0156 | HATA HATB HATC | Human Adrenal Gland |
| | | Tumor |
| H0163 | HSNA HSNB HSNC HSNM HSNN | Human Synovium |
| H0166 | HPEB HPEC | Human Prostate Cancer, |
| | | stage B2, frac. II |
| H0170 | HE2A HE2D HE2E HE2H HE2I HE2M | 12 Ek Old Early Stage |
| | | Human |
| H0175 | HASB | H. Adult Spleen, ziplox |
| H0179 | HNEA HNEB HNEC | Human Neutrophil |
| H0188 | HBNA HBNB | Human Normal Breast |
| H0212 | HPRT | Human Prostate, |
| | | subracted |
| H0218 | HT1S | Activated T-Cells, |
| | | Ohrs, subtracted |
| H0250 | HMQA | Human activated |
| | | monocytes |
| H0251 | HCDA HCDB HCDC HCDD HCDE | Human Chondrosarcoma |
| H0253 | HTLA HTLB HTLC | Human adult testis, |
| | | large inserts |
| H0254 | HLMA HLMC | BREAST LYMPH NODE |
| H0257 | H6EA | HL-60, PMA 4h |
| H0261 | HCER HCEV HCEW HCEX HCEY HCEZ | Human Cerebelllum- |
| | | Enzyme Subtracted |
| H0263 | HCQA HCQB | Human colon cancer |
| H0264 | HTOA HTOD HTOE HTOF HTOG | HUMAN TONSILS |
| H0265 | HTXA HTXB HTXC | ACTIVATED T-CELL (12 |
| | | hrs)/THIOURIDINE |
| | | LABELED (4 hrs) |
| H0266 | HMEA HMEC HMED HMEE | Human Microvascular |
| | | Endothelial Cells, |
| | | Fract. A |
| H0267 | HMEB | Human Microvascular |
| | | Endothelial Cells, |
| | | fract. B |
| H0288 | HUSA HUSC | Human Umbilical Vein |
| | | Endothelial Cells, |
| | | fract. A |
| H0269 | HUSB | Human Umbilical Vein |
| | | Endothelial Cells, |
| | | fract. B |
| H0270 | HPAS | HPAS (human pancreas, |
| | | subtracted) |
| H0271 | HNFA HNFB HNFC HNFD | Human Neutrophil, |
| | | Activated |
| H0272 | HTOB HTOC | HUMAN TONSILS, FRACTION |
| | | 2 |
| H0274 | HASC | Human Adult Spleen, |
| | | fraction II |
| H0275 | HIAT | Human Infact Adrendal |
| | | Gland, Subtracted |
| H0279 | HKEA | K562 cells |
| H0280 | HKFA | K562 + PMA (36 hrs) |
| H0281 | HLOA | Lymph node, abnorm. |
| | | cell line (ATCC #7225) |
| H0284 | HMGB | Human OB MG63 control |
| | | fraction I |
| H0286 | HMHB | Human OB MG63 treated |
| | | (10 nM E2) fraction I |
| H0288 | HOFB | Human OB HOS control |
| | | fraction I |
| H0290 | HOOB | Human OB HOS treated (1 |
| | | nM E2) fraction I |
| H0292 | HORB | Human OB HOS treated |
| | | (10 nM E2) fraction I |
| H0293 | HWIA | WI? |
| H0294 | HAUA HAUB | Amniotic cells - TNF |
| | | induced |
| H0295 | HAQA HAQB HAQC HAQD | Amniotic cells - |
| | | primary culture |
| H0300 | HCVA | CD34 positive cells |
| | | (Cord Blood) |
| H0305 | HCWA HCWB | CD34 positive cells |
| | | (Cord Blood) |
| H0306 | HCUA | CD34 depleted Buffy |
| | | Coat (Cord Blood) |
| S0001 | HFXA | Human Frontal Cortex |
| S0002 | HMSA HMSB HMSC HMSD HMSE HMSF | Human Monocytes, |
| | | Stimulated |
| S003 | HOSA HOSB HOSC HOSD | Human Osteoclastoma |
| S0007 | HEBA HEBB HEBC HEBD HEBE HEBN | EARLY STAGE HUMAN BRAIN |
| S0010 | HAGA HAGB HAGC | Human Amygdala |
| S0011 | HSRA HSRB HSRE | Human Osteoclastoma |
| | | Stromal Cells |
| S0022 | HSRD HSRF HSRG HSRH | Human Osteoclastoma |
| | | Stromal Cells |
| S0026 | HSQA HSQB HSQC HSQD HSQE HSQF | Stromal Cell Line TF274 |
| S0027 | HSKA HSKB HSKC | Human Smooth Muscle, |
| | | Serum Treated |
| S0028 | HSLA HSLB HSLC | Human Smooth Muscel, |
| | | Control |
| S0029 | HBHA | HUMAN BRAIN STEM |
| S0030 | HBPA | HUMAN BRAIN PONS |
| S0031 | HSDA | HUMAN SPINAL CORD |
| S0032 | HSJA HSJB HSJC | Human Smooth muscle |
| | | cells 1L-1B induced |
| S0035 | HBEA | Human Brain Medulla |
| S0036 | HSXA | Human Substantia Nigra |
| S0037 | HSHA HSHB HSHC | Human Smooth Muscle |
| | | Cells-PDGF induced |

Computational Analysis of ESTs and Databasing

The relational database management software Sybase has been used to construct a custom, specialized database for track information on the source and analysis of EST sequence data (Kerlavage, A. R., Adams, M. D., Kelley, J. C., Dubnick, M., Powell, J., Shanmugam, P., Venter, J. C., and Fields, C. 1993. Analysis and management of data from high-througput expressed sequence tag projects. Proceedings of the 26th Annual Hawaii International Conference on System Sciences, I:585-594). Tables in the database store information on the library, template prep and reaction protocols used for a particular sequence, and results of all the sequence analysis programs. An extensive set of computer programs has been developed to facilitate high-throughput analysis of EST sequences to provide completeness and consistency to the handling of sequence data and putative identifications. All new EST sequences are compared first to a set of known sequences that can be annotated automatically. This prescreen identifies mitochondrial and ribosomal RNA sequenes, several repetitive elements, and certain common sequences such as elongation factor 1 alpha in brain or gamma globin in fetal spleen. In general, matches between ESTs and database sequences cannot be annotated automatically. We use both BLAST (Altschul, 1990) and BLAZE (Intelligenetics, Inc.) to compare ESTs against the public databases.

All ESTs are compared at the nucleotide sequence level to GenBank and EMBL. All ESTs are also translated into the six possible peptide translations (three for each strand) and compared against Gen-Pept, Swiss-Prot and Protein Information Resource (PIR). The nucleotide sequence comparisons serve to identify exact matches to previously sequenced human genes and to distinguish between known genes and new, closely-related members of gene families. ESTs in the sequence listing of this application have no exact matches to sequences in the public databases. Peptide searches are much more sensitive in detecting relationships with genes from distantly related organisms and relatively degenerate protein motifs. Between fifteen and fifty percent of EST sequences can be identified based on the results of database searches. This broad variation is due to the several factors including the complexity of the library and the proportion of clones with coding sequence at the 5′ end. We have found that about half of the protein-coding ESTs have matches in the peptide databases; therefore, if all ESTs were protein-coding, half could be putatively identified based on similarity to sequences in the public databases.

The ESTs from all of the clones sequenced in this Example are identified herein as SEQ ID NOs 1-7483, set forth in the Sequence Listing, below.

### EXAMPLE 2

### EST Characterization

The EST sequences were initially examined for similarities in nucleotide and peptide databases. The nucleotide databases are: GenBank (GB) , and EMBL (E) ; the peptide databases are: GenPept (GP), Swiss-Prot (SP), and Protein Information Resource (PIR).

ESTs without exact Genbank matches were translated in all six reading frames and each translation was compared with the protein sequence database PIR and the ProSite protein motif database. Comparisons with the ProSite motif database were done by means of the program MacPattern from the EMBL Data Library. GenBank and PIR searches were conducted with the “basic local alignment search tool” programs for nucleotide (BLASTN) and peptide (BLASTX) comparisons (Altschul et al, J. Mol. Biol., 215:403 (1990)). PIR searches were run on the National Center for Biotechnology Information BLAST network service. The BLAST programs contain a very rapid database-searching algorithm that searches for local areas of similarity between two sequences and then extends the alignments on the basis of defined match and mismatch criteria. The algorithm does not consider the potential gaps to improve the alignment, thus sacrificing some sensitivity for a 6-80 fold increase in speed over other database-searching programs such as FASTA (Peqarson and Lipman, Proc. Natl. Acad. Sci. USA, 85:2444 (1988)).

Sequence similarities identified by the BLAST programs were considered statistically significant with a Poisson Pvalue less than 0.01. The Poisson P-value is the probability of as high a score occurring by chance given the number of residues in the query sequence and the database. After the BLASTN search, 30 unmatched ESTs were compared against GenBank by FASTA to determine if significant matches were missed due to the use of BLASTN for the database search. No additional statistically significant matches were found. Statistical significance does not necessarily mean functional similarity; some of the reported matches may indicate the presence of a conserved domain or motif or simply a common protein structure pattern. Those ESTs identified as fully corresponding to known human genes or proteins are not included in this disclosure.

The quality of the match is given as percent identity and length in base pairs for nucleotide matches and amino acid residues for peptide matches. In many cases ESTs match multiple domains on several related proteins.

The great majority of the partial cDNA sequences reported in Example 1 are unrelated to any sequences previously described in the literature.

Database entries in Table 2 include information regarding EST Identifier (EST).

In Table 2, the first seven characters of the EST identify the EST. EST's identified by the same first seven characters are obtained from the same clone. The last letter of the EST which is either “F” or “R” identifies the direction of sequencing, with “F” representing sequencing from the 3′ end and “R” sequencing from the 5′ end for all clones, except those identified initially with the letters HFK where the opposite is true.

Each EST is contained in a separate clone which is also identified by the identifier of Table 2 for the EST.

As hereinabove indicated, each clone has been partially sequenced, and such partial sequence is provided in the accompanying sequence list.

### EXAMPLE 3

### Isolation of a Selected Clone from the Deposited cDNA Library

Two approaches are used to isolate a particular clone out of the deposited cDNA library.

In the first, a clone is isolated directly by screening the library using an oligonucleotide probe. To isolate a particular clone, a specific oligonucleotide with 30-40 nucleotides is synthesized using an Applied Biosystems DNA synthesizer according to the EST sequence reported. The oligonucleotide is labeled with ³²P-ATP using T4 polynucleotide kinase and purified according to the standard protocol (Maniatis et al., Molecular Cloning: A Laboratory Manual, Cold Spring Harbor Press, Cold Spring, N.Y., 1982). The Lambda cDNA library deposited is be plated on 1.5% agar plate to the density of 20,000-50,000 pfu/150 mm plate. These plates are screened using Nylon membranes according to the standard phage screening protocol (Stratagene, 1993). Specifically, the Nylon membrane with denatured and fixed phage DNA is prehybridized in 6×SSC, 20 mM NaH₂PO₄, 0.4%SDS, 5×Denhardt's 500 μg/ml denatured, sonicated salmon sperm DNA; and 6×SSC, 0.1% SDS. After one hour of prehybridization, the membrane is hybridized with hybridization buffer 6×SSC, 20 mM NaH₂PO₄, 0.4% SDS, 500 ug/ml denatured, sonicated salmon sperm DNA with 1×10⁶ cpm/ml ³²P-probe overnight at 42° C. The membrane is washed at 45-50° C. with washing buffer 6×SSC, 0.1% SDS for 20-30 minutes dried and exposed to Kodak X-ray film overnight. Positive clones are isolated and purified by secondary and tertiary screening. The purified clone is be sequenced to verify its identity to the reported EST sequence.

An alternative approach to screen the deposited cDNA library is to prepare a DNA probe corresponding to the entire EST sequence. To prepare an EST probe, two oligonucleotide primers of 17-20 nucleotides derived from both ends of the EST sequence reported are synthesized and purified. These two oligonucleotide are used to amplify the EST probe using the cDNA library template. The DNA template is prepared from the phage lysate of the deposited cDNA library according to the standard phage DNA preparation protocol (Maniatis et al.). The polymerase chain reaction is carried out in 25 82 l of reaction mixture with 0.5 ug of the above cDNA template. The reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 min; elongation at 72° C. for 1 min) are performed with the Perkin-Elmer Cetus automated thermal cycler. The amplified product is analyzed by agarose gel electrophoresis and the DNA band with expected molecular weight is excised and purified. The PCR product is verified to be the EST probe by subcloning and sequencing the DNA product. The EST probe is labeled with the Multiprime DNA Labelling System (Amersham) at a specific activity <1×10⁹ dpm/μg. This probe is used to screen the deposited lambda cDNA library according to Stratagene's protocol. Hybridization is carried out with 5×TEN (20×TEN:0.3M Tris-HCl pH 8.0, 0.02M EDTA and 3M NaCl), 5×Denhardts, 0.5% sodium pyrophosphate, 0.1% SDS, 0.2 mg/ml heat denatured salmon sperm DNA and 1×10⁶ cpm/ml of [³²P]-labeled EST probe at 55° C. for 12 hours. The filters are washed in 0.5×TEN at room temperature for 20-30 min., then at 55° C. for 15 min. The filters are dried and autoradiographed at −70° C. using Kodak XAR-5 film. The positive clones are purified by secondary and tertiary screening. The sequence of the isolated clone are verified by DNA sequencing.

General procedures for obtaining complete sequences from ESTs are summarized as follows:

Procedure 1

Selected human DNA from an EST clone (the cDNA clone that was sequenced to give the EST), is purified e.g., by endonuclease digestion using EcoR1, gel electrophoresis, and isolation of the clone by removal from low melting agarose gel. The isolated insert DNA, is radiolabeled e.g., with 32P labels, preferably by nick translation or random primer labeling. The labeled EST insert is used as a probe to screen a lambda phage cDNA library or a plasmid cDNA library. Colonies containing clones related to the probe cDNA are identified and purified by known purification methods. The ends of the newly purified clones are nucleotide sequenced to identify full length sequences. Complete sequencing of full length clones is then performed by Exonuclease III digestion or primer walking. Northern blots of the mRNA from various tissues using at least part of the EST clone as a probe can optionally be performed to check the size of the mRNA against that of the purported full length cDNA.

The following procedures 2 and 3 can be used to obtain full length genes or full length coding portions of genes where a clone isolated from the deposited library does not contain a full length sequence. It is also applicable to obtaining full length sequences from clones obtained from sources other than the deposited library by use of the ESTs of the present invention.

Procedure 2

RACE Protocol for Recovery of Full-Length Genes

Partial cDNA clones can be made full-length by utilizing the rapid amplification of cDNA ends (RACE) procedure described in Frohman, M. A., Dush, M. K. and Martin, G. R. (1988) Proc. Nat'l. Acad. Sci. USA, 85:8998-9002. A cDNA clone missing either the 5′ or 3′ end can be reconstructed to include the absent base pairs extending to the translational start or stop codon, respectively. In most cases, cDNAs are missing the start of translation, therefor. The following briefly describes a modification of this original 5′ RACE procedure. Poly A+ or total RNA is reverse transcribed with Superscript II (Gibco/BRL) and an antisense or complementary primer specific to the cDNA sequence. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the da-tail in PCR buffer, Taq DNA polymerase (Perkin-Elmer Cetus), an oligo-dT primer containing three adjacent restriction sites (XhoI, SalI and ClaI) at the 5′ end and a primer containing just these restriction sites. This double-stranded cDNA is PCR amplified for 40 cycles with the same primers as well as a nested cDNA-specific antisense primer. The PCR products are size-separated on an ethidium bromide-agarose gel and the region of gel containing cDNA products the predicted size of missing protein-coding DNA is removed. cDNA is purified from the agarose with the Magic PCR Prep kit (Promega), restriction digested with XhoI or SalI, and ligated to a plasmid such as pBluescript SKII (Stratagene) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone.

Several quality-controlled kits are available for purchase. Similar reagents and methods to those above are supplied in kit form from Gibco/BRL. A second kit is available from Clontech which is a modification of a related technique, SLIC (single-stranded ligation to single-stranded cDNA), developed by Dumas et al. (Dumas, J. B., Edwards, M., Delort, J. and Mallet, J., 1991, Nucleic Acids Res., 19:5227-5232). The major differences in procedure are that the RNA is alkaline hydrolyzed after reverse transcription and RNA ligase is used to join a restriction site-containing anchor primer to the first-strand cDNA. This obviates the necessity for the dA-tailing reaction which results in a polyT stretch that is difficult to sequence past.

An alternative to generating 5′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

Procedure 3

RNA Ligase Protocol for Generating the 5′ End Sequences to Obtain Full Length Genes

Once a gene of interest is identified, several methods are available for the identification of the 5′ or 3′ portions of the gene which may not be present in the original EST clone. These methods include but are not limited to filter probing, clone enrichment using specific probes and protocols similar and identical to 5′ and 3′RACE. While the full length gene may be present in the library and can be identified by probing, a useful method for generating the 5′ end is to use the existing sequence information from the original EST to generate the missing information. A method similar to 5′RACE is available for generating the missing 5′ end of a desired full-length gene. (This method was published by Fromont-Racine et al., Nucleic Acids Res., 21(7):1683-1684 (1993). Briefly, a specific RNA oligonucleotide is ligated to the 5′ ends of a population of RNA presumably containing full-length gene RNA transcript and a primer set containing a primer specific to the ligated RNA oligonucleotide and a primer specific to a known sequence (EST) of the gene of interest, is used to PCR amplify the 5′ portion of the desired full length gene which may then be sequenced and used to generate the full length gene. This method starts with total RNA isolated from the desired source, poly A RNA may be used but is not a prerequisite for this procedure. The RNA preparation may then be treated with phosphatase if necessary to eliminate 5′ phosphate groups on degraded or damaged RNA which may interfere with the later RNA ligase step. The phosphatase if used is then inactivated and the RNA is treated with tobacco acid pyrophosphatase in order to remove the cap structure present at the 5′ ends of messenger RNAS. This reaction leaves a 5′ phosphate group at the 5′ end of the cap cleaved RNA which can then be ligated to an RNA oligonucleotide using T4 RNA ligase. This modified RNA preparation can then be used as a template for first strand cDNA synthesis using a gene specific oligonucleotide. The first strand synthesis reaction can then be used as a template for PCR amplification of the desired 5′ end using a primer specific to the ligated RNA oligonucleotide and a primer specific to the known sequence (EST) of the gene of interest. The resultant product is then sequenced and analyzed to confirm that the 5′ end sequence belongs to the EST.

### EXAMPLE 4

### Mapping of ESTs to Human Chromosomes

Randomly selected ESTs are assigned to chromosomes via PCR. Oligonucleotide primer pairs are designed from EST sequences to minimize the chance of amplifying through an intron. The oligonucleotides were 18-23 bp in length and designed for PCR amplification using the computer program INTRON (National Institutes of Mental Health, Bethesda, Md.) The program is based on the assumptions that: (1) introns are genomic sequences that interrupt the coding and noncoding sequences of genes (Smith, J. Mol. Evol., 27:45-55 (1988)); (2) there are consensus sequences for splice junctions (Shapiro, et al., Nucl. Acids Res., 15:7155-7174 (1987)); and (3) that 90% of the human genes studied have 3′ untranslated regions of mRNA not interrupted by introns in the genomic DNA (Hawkins, Nucl. Acids Res., 16:9893-9908 (1988)).

The program evaluates the likelihood that a given GG or CC dinucleotide represents a former exon-intron boundary. Specifically, every input strand is processed by the INTRON program twice, first evaluating the sense mRNA strand, and then processing the complementary or antisense strand. The program evaluates each sequence by finding all GG or CC pairs (possible former splice sites), searching for stop codons in all three reading frames, and analyzing the GG or CC pairs surrounded by stop codons. All regions of the EST that are unlikely to contain splice junctions based on CC content, GG content, and stop codon frequency are then marked by the program in uppercase.

The creation of PCR primers from known sequences is well known to those with skill in the art. For a review of PCR technology see Erlich, H. A., PCR Technology; Principles and Applications for DNA Amplification. 1992. W. H. Freeman and Co., New York. ESTs are examined for the presence of stop codons in each reading frame and for consensus splice junctions. The presence of stop codons and absence of splice junction sequences are more characteristic of 3′ untranslated sequences than of introns. The untranslated sequences are unique to a given gene; thus, primers from these regions are less likely to prime other members of a gene family or pseudogenes.

The primers are used in polymerase chain reactions (PCR) to amplify templates from total human genomic DNA. PCR conditions used are as follows: 60 ng of genomic DNA as a template for PCR with 80 ng of each oligonucleotide primer, 0.6 unit of Taq polymerase, and 1 uCu of a ³²P-labeled deoxycytidine triphosphate. The PCR is performed in a microplate thermocycler (Techne) under the following conditions: 30 cycles of 94° C., 1.4 min; 55° C., 2 min; and 72° C., 2 min; with a final extension at 72° C. for 10 min. The amplified products are analyzed on a 6% polyacrylamide sequencing gel and visualized by autoradiography. If the size of the resulting product is equivalent to the EST from which the primers are derived, then the PCR reaction is repeated with DNA templates from two panels of human-rodent somatic cell hybrids; BIOS PCRable DNA (BIOS Corporation) and NIGMS Human-Rodent Somatic Cell Hybrid Mapping Panel Number 1 (NIGMS, Camden, N.J.).

PCR is used to screen a series of somatic cell hybrid cell lines containing defined sets of human chromosomes for the presence of a given EST. DNA is isolated from the somatic hybrids and used as starting templates for PCR reactions using the primer pairs from EST sequences selected above. Only those somatic cell hybrids with chromosomes containing the human gene corresponding to the EST will yield an amplified fragment. ESTs are assigned to a chromosome by analysis of the segregation pattern of PCR products from hybrid DNA templates. For a review of techniques and analysis of results from somatic cell gene mapping experiments. See Ledbetter et al., Genomics, 6:475-481 (1990). The single human chromosome present in all cell hybrids that give rise to an amplified fragment represents the chromosome containing that EST.

The foregoing techniques are used to further localize ESTs and their associated genes to precise locations onto chromosomes, using sublocalization techniques that employ somatic cell hybrids. ESTs are used as hybridization probes and mapped to other chromosomes using techniques disclosed in Example 5. Somatic cell hybrids are prepared that contained defined subsets of chromosomes. Methods for preparing and selecting somatic cell hybrids are known in the art. For a review of an exemplary procedure to generate somatic cell hybrids containing the short arm of human chromosome 6, see Zoghbi, et al., Genomics, 9(4):713-720 (1991). For a general review of somatic cell hybridization see Ledbetter et al. (supra). The hybrids are processed to obtain DNA and analyzed by PCR and by fluorescence in situ hybridization.

### EXAMPLE 5

### Alternative Technique for Mapping to Chromosomes Mapping of ESTs to Chromosomes Using Fluorescence in situ Hybridization

This technique is used to map an EST to a particular location on a given chromosome. Cell cultures, tissue, or whole blood are used to obtain chromosomes.

Whole blood (0.5 ml) is added to RPMI 1640 and incubated 96 hours in a 5% CO₂/37° C. incubator. Colcemide (0.05 μg/ml) is added to the culture one hour before harvest. Cells are collected and washed in PBS. The suspension is incubated with a hypotonic solution of KC1 added dropwise to reach a final volume of 5 ml. The cells are spun down and fixed by resuspending the cells in methanol and glacial acetic acid (3:1). The cell suspension is dropped onto glass slides and dried.

The slides are treated with RNase A and washed, then dehydrated in a series of increasing concentrations of ethanol.

The EST to be localized is nick-translated using fluorescently labeled nucleotide (Korenberg, Jr., et al., Cell, 53(3):391-400 (1988)). Following nick translation, unincorporated label is removed by spin dialysis through Sepharose. The probe is further extracted with phenolchloroform to remove additional protein. The chromosomes are denatured in formamide using techniques known in the art and the denatured probe is added to the slides. Following hybridization, the cells are washed. The slides are studied under a fluorescent microscope. For a review of the technique see Verma et al., Human Chromosomes: A Manual of Basic Techniques. Pergamon Press, NY (1988), which is hereby incorporated by reference. In addition, the chromosomes can be stained for G-banding or Q-banding using techniques known in the art.

### EXAMPLE 6

### Automated DNA Sequencing Accuracy

ESTs that match human sequences in GenBank are excellent tools for the analysis of the accuracy of doublestrand automated DNA sequencing. EST/GenBank matches were examined for the number of nucleotide mismatches and gaps required to achieve optimal alignment by the Genetics Computer Group (GCG) program BESTFIT (Devereux et al, Nucleic Acids Research, 12: 387 (1984)). The number of mismatches, insertions and deletions was counted for each hundred bases of the sequence (Table 3). As expected, the sequence quality was best closest to the primer and decreased rapidly after about 400 bases. The number of deletions and insertions relative to the GenBank reference sequence increased five- to ten-fold beyond 400 bases, while the number of mismatches doubled. The average accuracy rate for individual double-stranded sequencing runs was 98.7% to 400 bases. No analysis was performed to determine whether discrepancies were due to errors in the ESTs or errors in the Genbank sequences.

TABLE 3
Sequencing Accuracy
Gaps

| | | | | | # of Bases |
|---|---|---|---|---|---|
| Window | Mismatches | Insertions | Deletions | Accuracy | Aligned |
| 101-200 | 1.21 | 0.01 | 0.05 | 98.73 | 15,500 |
| 201-300 | 1.20 | 0.06 | 0.03 | 98.71 | 15,274 |
| 301-400 | 1.94 | 0.06 | 0.03 | 98.71 | 12,342 |
| >400 | 3.48 | 2.73 | 0.32 | 93.48 | 5,381 |

Types of sequencing errors are separated into mismatches of the EST sequence with respect to the database sequence, and gaps, which are divided into insertions and deletions relative to the control sequence. The number of errors per 100 aligned bases are given for each error type as is the overall accuracy (correct base calls) as a percentage. Up to 85 base pairs of polylinker sequence is removed from the beginning of each EST, therefore, accuracy measurements began at bp 101.

### EXAMPLE 7

### cDNA Libraries Generated from Specific Genomic DNA by Exon Expression & Amplification

Exon amplification is used to express potential exons from genomic DNA in a recombinant vector that contains some of the signals necessary for splicing. If an exon is present in the proper orientation in the vector, that exon will be spliced in a mammalian cell and will become part of the mRNA of that cell. The exon splice-product can be purified from other mRNA in the cell by conversion of the mRNA to cDNA and selective amplification of the recombinant splice-product cDNAs. Cosmid DNA from human chromosome 19ql3.3 is digested with BamHI or BamHI/BglII restriction enzymes. The fragments generated are collected and size specifically cloned into an expression vector (Buckler, et al. Proc. Nat'l. Acad. Sci. USA, 88:4005-4009 (1991)). After transfection by electroporation of these constructs into COS cells, RNA transcripts are generated using the SV40 early promoter and a polyadenylation signal derived from SV40 both present in the expression vector. When a fragment of genomic DNA contains an entire exon with flanking intron sequence in the sense orientation, the exon should be retained in the mature poly(A)+ cytoplasmic RNA. Therefore, the mRNA is used as template for cDNA synthesis using reverse transcriptase and vector-priming. Subsequently, the cDNAs are amplified by vector-priming using PCR. A fraction of this first PCR product is reamplified using internal vector-primers containing terminal cloning sites. These products are end-repaired with T4 DNA polymerase, digested with the appropriate restriction enzymes, gel purified and cloned into pBluescript vectors. The constructs are transfected into XL1-Blue competent cells and plated on LB/Xgal/IPTG/ampicillin plates. White colonies are selected and expanded to prepare DNA templates as described in Example 1. When multiple cosmids or YAC clones are used as the source DNA, a pool of specific expressed exons is obtained as a cDNA library.

### EXAMPLE 8

### PCR Amplification from Predicted Exons

Computational analyses can be applied to genomic DNA sequences to predict protein coding regions. The coding region prediction program CRM (E. Uberbacher and R. Mural, Proc. Natl. Acad. Sci. USA, 88:11261-5 (1991)) finds open reading frames and classifies them according to their probability of being coding regions. These regions are subsequently examined using the GM program (C. Fields and C. Soderlund, Comp. Applic. Biosci., 6:263, 1990), which predicts intron-exon structure. PCR primers are then designed to amplify the predicted exons and used to test human cDNA libraries (for example, fetal brain or placental libraries) for the presence of these putative exons using a PCR assay.

### EXAMPLE 9

### Complete Sequence of EST Clone Inserts

There are a number of methods known to those with skill in the art of molecular biology to obtain sequence information from the cDNAs corresponding to the EST sequences. Procedures for these methods are provided in Basic Methods in Molecular Biology (David et al. supra). One way to acquire more information about the cDNA from which an EST was derived is to sequence the remainder of the cDNA clone.

Briefly, EST clones are digested with the restriction enzymes SalI and KpnI or PstI and BamHI (for deletions from the Forward primer and Reverse primer ends of the insert, respectively). The KpnI and PstI enzymes leave 3′ sticky ends following digestion, which Exonuclease III is unable to bind. This results in unidirectional deletions into the cDNA insert leaving the vector sequence undisturbed. After addition of Exonuclease III to the Forward and Reverse deletion reactions, aliquots of the reaction are removed at defined time intervals and the reaction is stopped to prevent further deletion. S1 nuclease and Klenow DNA polymerase are added to create blunt ended fragments suitable for ligation. Samples for each time point are purified by electrophoresis through an agarose gel and religated. Two to four representative clones from each time point in each direction are sequenced to give between 200 and 400 base pairs of sequence data. Careful selection of deletion conditions and time points allow a deletion series of approximately 100-200 base pairs difference in length at each consecutive time point. Sequence fragments are reassembled into a redundant contiguous sequence using the INHERIT software from Applied Biosystems, Inc. (Foster City, Calif.) In this way, the complete insert from the cDNA clone is sequenced on both strands to an average redundancy between three and four (each base is sequenced between three and four times, on average).

### EXAMPLE 10

### Determining Reading Frame, Orientation, Coding Regions: ESTs and Complete cDNA Sequences

Once the complete cDNA sequence has been determined in accordance with Example 9, the reading frame, orientation, and coding regions are determined by computer techniques. (The complete coding region is considered to be the largest open reading frame from a methionine to a stop codon.)

Specifically, the CRM program on the GRAIL server is used to determine probable coding regions. This information is supplemented by location of start and stop codons. Where possible, the results of the CRM analysis are validated by comparison of the cDNA sequence to known sequences using database matching, in accordance with Example 2. If a match of 50% (or even less) is found in any particular reading frame and orientation, this serves to verify corresponding CRM results. Alternatively, database matches can be used to determine reading frame and orientation without use of the CRM program, of course, if the cDNA is derived from a directional library, the probable orientation is already known.

### EXAMPLE 11

### Preparation of PCR Primers and Amplification of DNA

The EST sequences and the corresponding cDNA sequences and genomic sequences can be used, in accordance with the present invention, to prepare PCR primers for a variety of uses. The PCR primers are preferably at least 15 bases, and more preferably at least 18 bases in length. The procedure of Example 3 is repeated using the desired EST, or using the corresponding cDNA or genomic DNA sequence from Example 10. It is preferred that the primer pairs have approximately the same G/C ratio, so that melting temperatures are approximately the same. When screening cDNA, introns are of no concern; however, when screening genomic DNA, primers should be selected to avoid reading across introns, which usually are too large to amplify. The PCR primers and amplified DNA of this Example find use in the Examples that follow.

### EXAMPLE 12

### Forensic Matching by DNA Sequencing

In one exemplary method, DNA samples are isolated from forensic specimens of, for example, hair, semen, blood or skin cells by conventional methods. A panel of PCR primers derived from a number of the sequences of Example 1, 9, 10 and/or 11 is then utilized in accordance with Example 10 to obtain DNA of approximately 100-200 bases in length from the forensic specimen. Corresponding sequences are obtained from a suspect. Each of these identification DNAs is then sequenced, and a simple database comparison determines the differences, if any, between the sequences from the suspect and those from the sample. Statistically significant differences between the suspect's DNA sequences and those from the sample conclusively prove a lack of identity. This lack of identity can be proven, for example, with only one sequence. Identity, on the other hand, should be demonstrated with a large number of sequences, all matching. Preferably, a minimum of 50 statistically identical sequences of 100 bases in length are used to prove identity between the suspect and the sample.

### EXAMPLE 13

### Positive Identification by DNA Sequencing

The technique outlined in the previous example may also be used on a larger scale to provide a unique fingerprinttype identification of any individual. In this technique, primers are prepared from a large number of sequences from Examples 1, 7, 8 and/or 9. Preferably, 20 to 50 different primers are used. These primers are used to obtain a corresponding number of PCR-generated DNA segments from the individual in question in accordance with Example 11. Each of these DNA segments is sequenced, using the methods set forth in Example 1. The database of sequences generated through this procedure uniquely identifies the individual from whom the sequences were obtained. The same panel of primers may then be used at any later time to absolutely correlate tissue or other biological specimen with that individual.

### EXAMPLE 14

### Southern Blot Forensic Identification

The procedure of Example 13 is repeated to obtain a panel of from 10 to 2000 amplified sequences from an individual and a specimen. This PCR-generated DNA is then digested with one or a combination of, preferably, four base specific restriction enzymes. Such enzymes are commercially available and known to those of skill in the art. After digestion, the resultant gene fragments are size separated in multiple duplicate wells on an agarose gel and transferred to nitrocellulose using Southern blotting techniques well known to those with skill in the art. For a review of Southern blotting see Davis et al. (Basic Methods in Molecular Biology, 1986, Elsevier Press. pp 62-65).

A panel of ESTs or complete cDNA sequences from Examples 1, and/or 9, or fragments thereof of at least 15 bases, are radioactively or colorimetrically labeled using end-labeled oligonucleotides derived from the ESTs, nick translated sequences or the like using methods known in the art and hybridized to the Southern blot using techniques known in the art (Davis et al., supra). Preferably, at least 5 to 10 of these labeled probes are used, and more preferably at least about 20 or 30 are used to provide a unique pattern. The resultant bands appearing from the hybridization of a large sample of ESTs will be a unique identifier. Since the restriction enzyme cleavage will be different for every individual, the band pattern on the Southern blot will also be unique. Increasing the number of EST probes will provide a statistically higher level of confidence in the identification since there will be an increased number of sets of bands used for identification.

### EXAMPLE 15

### Dot Blot Identification Procedure

Another technique for identifying individuals using the sequences disclosed herein utilizes a dot blot hybridization technique.

Genomic DNA is isolated from cell nuclei of subjects to be identified. Oligonucleotide probes of approximately 30 bp in length are synthesized that correspond to sequences from the ESTs. The probes are used to hybridize to the genomic DNA under conditions known to those in the art. The oligonucleotides are end labelled with ³²P using polynucleotide kinase (Pharmacia). Dot blots are created by spotting about 50 ng cDNA of at least 10, preferably at least 50 sequences corresponding to a variety of the Sequence ID NOs provided in Table 4 onto nitrocellulose or the like using a vacuum dot blot manifold (BioRad, Richmond Calif.). The nitrocellulose filter containing the EST clone sequences is baked or UV linked to the filter, prehybridized and hybridized with labeled probe using techniques known in the art (Davis et al., supra). The ³²P labeled DNA fragments are sequentially hybridized with successively stringent conditions to detect minimal differences between the 30 bp sequence and the DNA. Tetramethylammonium chloride is useful for identifying clones containing small numbers of nucleotide mismatches (Wood et al., Proc. Natl. Acad. Sci. USA 82(6):1585-1588 (1985) which is hereby incorporated by reference. A unique pattern of dots distinguishes one individual from other individuals.

### EXAMPLE 16

### Alternative “Fingerprint” Identification Technique

EST sequences and the corresponding complete cDNA sequences can be used to create a unique fingerprint for an individual. Thus pools of EST sequences can be used in forensics, paternity suits or the like to differentiate one individual from another.

Entire EST sequences can be used; similarly oligonucleotides can be prepared from EST sequences. In this example, 20-mer oligonucleotides are prepared from 200 EST sequences using commercially available oligonucleotide services such as Oligos Etc., Wilsonville, Oreg. Patient cell samples are processed for DNA using techniques well known to those with skill in the art. The nucleic acid is digested with restriction enzymes EcoRI and XbaI. Following digestion, samples are applied to wells for electrophoresis. The procedure, as known in the art, can be modified to accommodate polyacrylamide electrophoresis, however in this example, samples containing 5 μg of DNA are loaded into wells and separated on 0.8% agarose gels. The gels are transferred using Southern blotting techniques onto nitrocellulose.

10 ng of each of the oligos are pooled and end-labeled with 32P. The nitrocellulose is prehybridized with blocking solution and hybridized with the labeled probes. Following hybridization and washing, the nitrocellulose filter is exposed to X-Omat AR X-ray film. The resulting hybridization pattern will be unique for each individual.

It is additionally contemplated within this example that the representative number of EST sequences can be varied for additional accuracy or clarity.

### EXAMPLE 17

### Identification of Genes Associated with Hereditary Diseases

This example illustrates an approach useful for the association of EST sequences with particular phenotypic characteristics. In this example, a particular EST is used as a test probe to associate that EST with a particular phenotypic characteristic.

Cells from patients with these diseases are isolated and expanded in culture. PCR primers from the EST sequences are used to screen genomic DNA and RNA or cDNA from the patients. ESTs that are not amplified in the patients can be positively associated with a particular disease by further analysis.

### EXAMPLE 18

### Identification of a Gene Associated with Angelman's Disease

This example illustrates the manner in which EST's can be used to identify gene(s) associated with a disease. The technique is described with respect to Angelman's disease; however, the technique is generally applicable to other diseases.

Angelman's disease (AD) is characterized by deletions on the long arm of chromosome 15 (15q11q13) (Williams et al. Am. J. Med. Genet. 32:339-345 (1989) hereby incorporated by reference). The symptoms of the disease include developmental delay, seizures, inappropriate laughter and ataxic movements. These symptoms suggest that the disorder is a neurologic deficiency. This example illustrates how ESTs may be used in identifying the defective gene or genes associated with Angelman's Disease. (The example is based on analogous work with genomic DNA, rather than cDNA and ESTs, in identifying the genetic defect associated with Angelman's Disease.) This example is generally applicable to the use of how EST sequences may generally be used for identifying gene sequences associated with an inherited disease that is mapped to a chromosome location.

ESTs are screened using techniques described in Example 3 and Example 5 to identify those ESTs that localize to the long arm of chromosome 15 and preferably localize to chromosome 15 bands 15q11q13 from normal patients. ESTs that bind to the long arm of chromosome 15 are hybridized to chromosome 15 from AD patients. These studies are preferably performed using either fluorescence in situ hybridization or using somatic cell hybrids that contain fragments from the long arm of chromosome 15 from AD patients. Those chromosome 15-specific ESTs that do not map to chromosome 15 from AD patients are useful as markers for Angelman's Disease and can be incorporated into diagnostics for genetic screening. These ESTs are associated with chromosome deletions present in Angelman's disease. Identification of the gene associated with these AD negative ESTs and an analysis of the-polypeptides encoded by the genes from normal patients is essential for providing gene, or other therapies for AD patients.

Genetic diseases are not always accompanied by gene deletions. Therefore, it is also important to use the ESTs that bind to bands 15q11q13 from AD patients as tools to identify the polymorphisms present within the disease population. Restriction fragment length polymorphism (RFLP) analysis can be performed on patient cells from AD disease or from somatic cell hybrids created using the long arm of chromosome 15. For a review of RFLP techniques see DonisKeller et al. (Cell, 51:319-337 (1987) hereby incorporated by reference) . DNA is isolated from the somatic cell lines or from cells from AD patients. The DNA is digested with one or more restriction enzymes according to techniques of Donis-Keller et al. The resulting fragments are separated by gel electrophoresis, denatured, transferred to nitrocellulose and hybridized with the selected radiolabeled ESTs that localize to the region of interest. The autoradiographic pattern is compared both to a number of AD patients and to normal patients. Common patterns of EST hybridization in AD patients that are not present in normal patients indicates that the genes associated with these ESTs are candidate genes affected by AD.

cDNA libraries are prepared from the somatic cell hybrids from AD patients. Libraries are prepared using Lambda Zap II Library Kits (Stratagene, La Jolla, Calif.) or other commercially available library kits. The ESTs of interest are used as probes to identify those colonies carrying genes corresponding to the EST probes. Positive clones are sequenced and the sequences are compared to homologous gene sequences derived from normal patients.

Alterations, including deletions and substitutions, within gene sequences, associated with bands 15q11q13, are thus positively identified and associated with AD disease. Wagstaff et al. were able to identify deletions and substitutions in sequences encoding the GABA receptor protein subunit from patients with Angelman's disease (Am. J. Hum. Genet. 49:330-337, (1991)). It is likely that other genes will additionally be associated with the disease.

### EXAMPLE 19

### Preparation and Use of Antisense Oligonucleotides

Antisense RNA molecules are known to be useful for regulating translation within the cell. Antisense RNA molecules can be produced from EST sequences or from the corresponding gene sequences. These antisense molecules can be used as diagnostic probes to determine whether or not a particular gene is expressed in a cell. Similarly, the antisense molecules can be used as a therapeutic to regulate gene expression once the EST is associated with a particular disease (see Example 18).

The antisense molecules are obtained from a nucleotide sequence by reversing the orientation of the coding region with regard to the promoter. Thus, the antisense RNA is complementary to the corresponding mRNA. For a review of antisense design see Green et al., Ann. Rev. Biochem. 55:569-597 (1986), which is hereby incorporated by reference. The antisense sequences can contain modified sugar phosphate backbones to increase stability and make them less sensitive to RNase activity. Examples of the modifications are described by Rossi et al., Pharmacol. Ther. 50(2):245-254, (1991).

Antisense molecules are introduced into cells that express the gene corresponding to the EST of interest in culture. In a preferred application of this invention, the polypeptide encoded by the gene is first identified, so that the effectiveness of antisense inhibition on translation can be monitored using techniques that include but are not limited to antibody-mediated tests such as RIAs and ELISA, functional assays, or radiolabelling. The antisense molecule is introduced into the cells by diffusion or by transfection procedures known in the art. The molecules are introduced onto cell samples at a number of different concentrations preferably between 1×10⁻¹⁰M to 1×10⁻⁴M. Once the minimum concentration that can adequately control translation is identified, the optimized dose is translated into a dosage suitable for use in vivo. For example, an inhibiting concentration in culture of 1×10⁻⁷M translates into a dose of approximately 0.6 mg/kg body weight. Levels of oligonucleotide approaching 100 mg/kg body weight or higher may be possible after testing the toxicity of the oligonucleotide in laboratory animals.

The antisense molecules can be introduced into the body as an oligonucleotide, an oligonucleotide encapsulated in lipid, oligonucleotide sequence encapsidated by viral protein, or (as oligonucleotide contained in an expression vector such as those described in Example 21). The antisense oligonucleotide is preferably introduced into the vertebrate by injection. It is additionally contemplated that cells from the vertebrate are removed, treated with the antisense oligonucleotide, and reintroduced into the vertebrate. It is further contemplated that the antisense oligonucleotide sequence is incorporated into a ribozyme sequence to enable the antisense to bind and cleave its target. For technical applications of ribozyme and antisense oligonucleotides see Rossi et al.

### EXAMPLE 20

### Preparation and Use of Triple Helix Probes

Triple helix oligonucleotides are used to inhibit transcription from a genome. They are particularly useful for studying alterations in cell activity as it is associated with a particular gene. The EST sequences or complete sequences of the present invention or, more preferably, a portion of those sequences, can be used to inhibit gene expression in individuals having diseases associated with a particular gene. Similarly, a portion of the EST or corresponding gene sequence can be used to study the effect of inhibiting transcription of a particular gene within a cell. Traditionally, homopurine sequences were considered the most useful. However, homopyrimidine sequences can also inhibit gene expression. Thus, both types of sequences from either the EST or from the gene corresponding to the EST are contemplated within the scope of this invention. Homopyrimidine oligonucleotides bind to the major groove at homopurine:homopyrimidine sequences. As an example, 10-mer to 20-mer homopyrimidine sequences from the ESTs can be used to inhibit expression from homopurine sequences. Several of the EST sequences contain homopyrimidine 15-mers. Moreover the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For background information on the generation of oligonucleotides suitable for triple helix formation. See Griffin et al., Science, 245:967-971 (1989), which is hereby incorporated by this reference).

The oligonucleotides may be prepared on an oligonucleotide synthesizer or they may be purchased commercially from a company specializing in custom oligonucleotide synthesis. The sequences are introduced into cells in culture using techniques known in the art that include but are not limited to calcium phosphate precipitation, DEAE-Dextran, electroporation, liposome-mediated transfection or native uptake. Treated cells are monitored for altered cell function. These cell functions are predicted based upon the homologies of the gene, corresponding to the EST from which the oligonucleotide was derived, with known genes sequences—that have been associated with a particular function. The cell functions can also be predicted based on the presence of abnormal physiologies within cells derived from individuals with a particular inherited disease, particularly when the EST is associated with the disease using techniques described in this example.

### EXAMPLE 21

### Gene Expression from DNA Sequences Corresponding to ESTs

A gene sequence of the present invention coding for all or part of a human gene product is introduced into an expression vector using conventional technology. (Techniques to transfer cloned sequences into expression vectors that direct protein translation in mammalian, yeast, insect or bacterial expression systems are well known in the art.) Commercially available vectors and expression systems are available from a variety of suppliers including Stratagene (La Jolla, Calif.), Promega (Madison, Wis.), and Invitrogen (San Diego, Calif.). If desired, to enhance expression and facilitate proper protein folding, the codon context and codon pairing of the sequence may be optimized for the particular expression organism, as explained by Hatfield, et al., U.S. Pat. No. 5,082,767, incorporated herein by this reference.

The following is provided as one exemplary method to generate polypeptide(s) from cloned cDNA sequence(s) which include the coding region for the peptide of interest and which cDNA sequences are obtained by use of an EST of the present invention, as hereinabove described. If the cDNA lacks a poly A sequence, this sequence can be added to the construct by, for example, splicing out the poly A sequence from pSG5 (Stratagene) using BglI and SalI restriction endonuclease enzymes and incorporating it into the mammalian expression vector pXT1 (Stratagene). pXT1 contains the LTRs and a portion of the gag gene from Moloney Murine Leukemia Virus. The position of the LTRs in the construct allow efficient stable transfection. The vector includes the Herpes Simplex thymidine kinase promoter and the selectable neomycin gene. The cDNA is obtained by PCR from the bacterial vector using oligonucleotide primers complementary to the cDNA and containing restriction endonuclease sequences for PstI incorporated into the 5′ primer and BglII at the 5′ end of the corresponding cDNA 3′ primer, taking care to ensure that the cDNA is positioned such that its followed with the poly A sequence. The purified fragment obtained from the resulting PCR reaction is digested with PstI, blunt ended with an exonuclease, digested with BglII, purified and ligated to pXT1, now containing a poly A sequence and digested BglII.

The ligated product is transfected into mouse NIH 3T3 cells using Lipofectin (Life Technologies, Inc., Grand Island, N.Y.) under conditions outlined in the product specification. Positive transfectants are selected after growing the transfected cells in 600 ug/ml G418 (Sigma, St. Louis, Mo.). The protein is preferably released into the supernatant. However if the protein has membrane binding domains, the protein may additionally be retained within the cell or expression may be restricted to the cell surface.

Since it may be necessary to purify and locate the transfected product, synthetic 15-mer peptides synthesized from the predicted cDNA sequence are injected into mice to generate antibody to the polypeptide encoded by the cDNA.

If antibody production is not possible, the cDNA sequence is additionally incorporated into eukaryotic expression vectors and expressed as a chimeric with, for example, β-globin. Antibody to β-globin is used to purify the chimeric. Corresponding protease cleavage sites engineered between the β-globin gene and the cDNA are then used to separate the two polypeptide fragments from one another after translation. One useful expression vector for generating β-globin chimerics is pSG5 (Stratagene). This vector encodes rabbit β-globin. Intron II of the rabbit β-globin gene facilitates splicing of the expressed transcript, and the polyadenylation signal incorporated into the construct increases the level of expression. These techniques as described are well known to those skilled in the art of molecular biology. Standard methods are published in methods texts such as Davis et al. and many of the methods are available from the technical assistance representatives from Stratagene, Life Technologies, Inc., or Promega. Polypeptide may additionally be produced from either construct using in vitro translation systems such as In vitro Express™ Translation Kit (Stratagene)

### Example 22

### Production of an Antibody to a Human Protein

Substantially pure protein or polypeptide is isolated from the transfected or transformed cells as described in Example 21. The protein can also be produced in a recombinant prokaryotic expression system, such as E. coli, or can by chemically synthesized. Concentration of protein in the final preparation is adjusted, for example, by concentration on an Amicon filter device, to the level of a few micrograms/ml. Monoclonal or polyclonal antibody to the protein can then be prepared as follows:

A. Monoclonal Antibody Production by Hybridoma Fusion

Monoclonal antibody to epitopes of any of the peptides identified and isolated as described can be prepared from murine hybridomas according to the classical method of Kohler, G. and Milstein, C., Nature, 256:495 (1975) or modifications of the methods thereof. Briefly, a mouse is repetitively inoculated with a few micrograms of the selected protein over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol., 70:419 (1980), and modified methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2 (date ?)

B. Polyclonal Antibody Production by Immunization

Polyclonal antiserum containing antibodies to heterogenous epitopes of a single protein can be prepared by immunizing suitable animals with the expressed protein described above, which can be unmodified or modified to enhance immunogenicity. Effective polyclonal antibody production is affected by many factors related both to the antigen and the host species. For example, small molecules tend to be less immunogenic than other and may require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971).

Booster injections can be given at regular intervals, and antiserum harvested when antibody titer thereof, as determined semi-quantitatively, for example, by double inmunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D. , Chap. 42 in: Manual of Clinical Immunology, 2d Ed. (Rose and Friedman, eds.) Amer. Soc. For Microbiology, Washington, D.C. (1980).

Antibody preparations prepared according to either protocol are useful in quantitative immunoassays which determine concentrations of antigen-bearing substances in biological samples; they are also used semi-quantitatively or qualitatively to identify the presence of antigen in a biological sample.

### EXAMPLE 23

### Identification of Tissue Types or Cell Species by Means of Labeled Tissue Specific Antibodies

Identification of specific tissues is accomplished by the visualization of tissue specific antigens by means of antibody preparations according to Example 24 which are conjugated, directly or indirectly to a detectable marker. Selected labeled antibody species bind to their specific antigen binding partner in tissue sections, cell suspensions, or in extracts of soluble proteins from a tissue sample to provide a pattern for qualitative or semi-qualitative interpretation.

Antisera for these procedures must have a potency exceeding that of the native preparation, and for that reason, antibodies are concentrated to a mg/ml level by isolation of the gamma globulin fraction, for example, by ion-exchange chromatography or by ammonium sulfate fractionation. Also, to provide the most specific antisera, unwanted antibodies, for example to common proteins, must be removed from the gamma globulin fraction, for example by means, of insoluble immunoabsorbents, before the antibodies are labeled with the marker. Either monoclonal or heterologous antisera is suitable for either procedure.

A. Immunohistochemical Techniques

Purified, high-titer antibodies, prepared as described above, are conjugated to a detectable marker, as described, for example, by Fudenberg, H., Chap. 26 in: Basic & Clinical Immunology, 3rd Ed. Lange, Los Altos, Calif. (1980) or Rose, N. et al., Chap. 12 in: Methods in Immunodiagnosis, 2d Ed. John Wiley & Sons, New York (1980).

A fluorescent marker, either fluorescein or rhodamine, is preferred, but antibodies can also be labeled with an enzyme that supports a color producing reaction with a substrate, such as horseradish peroxidase. Markers can be added to tissue-bound antibody in a second step, as described below. Alternatively, the specific antitissue antibodies can be labeled with ferritin or other electron dense particles, and localization of the ferritin coupled antigen-antibody complexes achieved by means of an electron microscope. In yet another approach, the antibodies are radiolabeled, with, for example ¹²⁵I, and detected by overlaying the antibody treated preparation with photographic emulsion.

Preparations to carry out the procedures can comprise monoclonal or polyclonal antibodies to a single gene copy or protein, identified as specific to a tissue type, for example, brain tissue, or antibody preparations to several antigenically distinct tissue specific antigens can be used in panels, independently or in mixtures, as required.

Tissue sections and cell suspensions are prepared for immunohistochemical examination according to common histological techniques. Multiple cryostat sections (about 4 pm, unfixed) of the unknown tissue and known control, are mounted and each slide covered with different dilutions of the antibody preparation. Sections of known and unknown tissues should also be treated with preparations to provide a positive control, a negative control, for example, pre-immune sera, and a control for non-specific staining, for example, buffer.

Treated sections are incubated in a humid chamber for 30 min at room temperature, rinsed, then washed in buffer for 30-45 min. Excess fluid is blotted away, and the marker developed.

If the tissue specific antibody was not labeled in the first incubation, it can be labeled at this time in a second antibody-antibody reaction, for example, by adding fluorescein- or enzyme-conjugated antibody against the immunoglobulin class of the antiserum-producing species, for example, fluorescein labeled antibody to mouse IgG. Such labeled sera are commercially available.

The antigen found in the tissues by the above procedure can be quantified by measuring the intensity of color or fluorescence on the tissue section, and calibrating that signal using appropriate standards.

B. Identification of Tissue Specific Soluble Proteins

The visualization of tissue specific proteins and identification of unknown tissues from that procedure is carried out using the labeled antibody reagents and detection strategy as described for immunohistochemistry; however the sample is prepared according to an electrophoretic technique to distribute the proteins extracted from the tissue in an orderly array on the basis of molecular weight for detection.

A tissue sample is homogenized using a Virtis apparatus; cell suspensions are disrupted by Dounce homogenization or osmotic lysis, using detergents in either case as required to disrupt cell membranes, as is the practice in the art. Insoluble cell components such as nuclei, microsomes, and membrane fragments are removed by ultracentrifugation, and the soluble protein-containing fraction concentrated if necessary and reserved for analysis.

A sample of the soluble protein solution is resolved into individual protein species by conventional SDS polyacrylamide electrophoresis as described, for example, by Davis, L. et al., Section 19-2 in: Basic Methods in Molecular Biology (P. Leder, ed), Elsevier, New York (1986), using a range of amounts of polyacrylamide in a set of gels to resolve the entire molecular weight range of proteins to be detected in the sample. A size marker is run in parallel for purposes of estimating molecular weights of the constituent proteins. Sample size for analysis is a convenient volume of from 5-50 μl, and containing from about 1 to 100 μg protein. An aliquot of each of the resolved proteins is transferred by blotting to a nitrocellulose filter paper, a process that maintains the pattern of resolution. Multiple copies are prepared. The procedure, known as Western Blot Analysis, is well described in Davis, L. et al., (supra at Section 19-3). One set of nitrocellulose blots is stained with Coomassie Blue dye to visualize the entire set of proteins for comparison with the antibody bound proteins. The remaining nitrocellulose filters are then incubated with a solution of one or more specific antisera to tissue specific proteins. In this procedure, as in procedure A above, appropriate positive and negative sample and reagent controls are run.

In either procedure A or B, a detectable label can be attached to the primary tissue antigen-primary antibody complex according to various strategies and permutations thereof. In a straightforward approach, the primary specific antibody can be labeled; alternatively, the unlabeled complex can be bound by a labeled secondary anti-IgG antibody. In other approaches, either the primary or secondary antibody is conjugated to a biotin molecule, which can, in a subsequent step, bind an avidin conjugated marker. According to yet another strategy, enzyme labeled or radioactive protein A, which has the property of binding to any IgG, is bound in a final step to either the pritary or secondary antibody.

The visualization of tissue specific antigen binding at levels above those seen in control tissues to one or more tissue specific antibodies, prepared from the gene sequences identified from EST sequences, can identify tissues of unknown origin, for example, forensic samples, or differentiated tumor tissue that has metastasized to foreign bodily sites.

### EXAMPLE 26

### Identification of Tissue Types or Cell Species by Means of Labeled in situ Hybridization

The ESTs, full or partial coding length DNA sequences obtainable from the deposited material and unique DNA fragments of the DNA sequences which are nonoverlapping or fully or partially overlapping with the ESTs can be used in in situ hybridization diagnostic assay protocols for the deprotection of genetic anomalies or diseases, such as for example Huntington's Chorea. The level of detection sensitivity currently available in the in situ hybridization field using known labeling systems is as low as a single DNA copy in a single cell.

Cells from a patient whose tissue is to be analyzed are deposited either as tissue sections or as single cell suspensions on a solid support such as a glass slide and then fixed with a fixative that provides the best spatial resolution of the cells and the optimal hybridization efficiency. After fixation, the support bound cells can be dehydrated and stored at room temperature or the hybridization procedure can be carried out immediately.

The hybridization step uses, for example, an EST characteristic of the DNA sequence whose absence is associated with Huntington's chorea or involuntary tremor. Thus, the ESTs or other DNA sequence of the invention are used as a probe when appropriately labeled with an isotopic or nonisotopic label and placed in a hybridization solution containing prepared, for example, of concentrated SSC solution (1x=0.15M sodium chloride and 0.015M sodium citrate), a buffer such as 0.1M sodium phosphate (ph 7.4), approximately 100 micrograms/milliliter of a nonspecific low molecular weight DNA to diminish nonspecific binding, a detergent such as 0.1% Triton X-100 to facilitate probe entry into the cells and about 10-20 mM of vanadyl ribonucleoside complexes.

The hybridization solution containing the probe is pipetted or otherwise deposited onto the slide in an amount sufficient to cover the cells. The cells are then incubated at, for example, 55° C. for at least about 30 minutes. The probe is added at a high concentration, e.g. at least about 1 microgram/milliliter of hybridization mixture in order to give optimal results in the shortest time frame.

The ESTs can be directly labeled prior to addition to the hybridization solution or a secondary hybridization of the present invention between the sought for target DNA sequence having a label thereon can be used to “sandwiched” the DNA or RNA where present and the secondary label probe. Such detectable labels are well known -and include, for example, enzymes, enzyme substrates, coenzymes and enzyme inhibitors; chromofors, luminesce, luminofors such as chemilluminescers and bioluminescers; specifically bindable ligands; and isotopic ionic labels.

The hybridization of solution and inbound probe are washed from the slides and the specimens are analyzed by observation of cytomorphology as compared to fresh, untreated cells using a phase contrast microscope.

There are many methods available to hybridize labeled probes in solution to nucleic acids immobilized on slides. These methods differ in the following respects:

Solvent and temperature used (e.g., 68° C. in aqueous solution or 42° C. in 50% formamide);

Volume of solvent and length of hybridization (large volumes for periods as long as 3 days or minimal volumes for times as short as 4 hours);

Degree and method of agitation (continuous shaking or stationary);

Use of agents such as Denhardt's reagent to block the non-specific attachment of the probe to the surface of the solid matrix;

Concentration of the labeled probe and its specific activity;

Use of compounds, such as dextran sulfate (Wahl et al. 1979) or polyethylene glycol (Renz and Kurz 1984; Amasino 1986), that increase the rate of reassociation of nucleic acids; and

Stringency of washing following the hybridization.

Factors modified using conventional levels of skill include:

1. The smaller the volume of hybridization solution, the better. In small volumes of solution, the kinetics of nucleic acid reassociation are faster and the amount of probe needed can be reduced so that the DNA on the slide acts as the driver for the reaction. However, it is essential that sufficient liquid be present for the sample to remain covered at all times by a film of the hybridization solution.

2. Continual movement of the probe solution across the filter is unnecessary, even for a reaction driven by the DNA immobilized on the slide. However, if a large number of slides are hybridized simultaneously, agitation or mechanical separation is advisable to prevent the slides from adhering to one another.

3. Several different types of agents can be used to block the nonspecific attachment of the probe to the surface of the slide. These include Denhardt's reagent (Denhardt 1966), heparin, and nonfat dried milk (Johnson et al. 1984). Frequently, these agents are used in combination with denatured, fragmented salmon sperm or yeast DNA and detergents such as SDS. Virtually complete suppression of background hybridization is obtained by prehybridizing with a blocking agent consisting of 5×Denhardt's reagent, 0.5% SDS, and 100 μg/ml denatured, fragmented DNA. This mixture is particularly desirable whenever the signal-to-noise ratio is expected to be low, for example, when carrying out Northern analysis of low-abundance mRNAs or Southern hybridizations with single-copy sequences of mammalian DNA.

4. To maximize the rate of annealing of the probe with its target, hybridizations are usually carried out in solutions of high ionic strength (6×SSC or 6×SSPE) at a temperature that is 20-25° C. below the melting temperature (Tₘ). Both solutions work equally well when hybridization is carried out in aqueous solvents. However, formamide is included in the hybridization buffer, 6×SSPE is preferred because of its greater buffering power.

5. In general, the washing conditions should be as stringent as possible (i.e., a combination of temperature and salt concentration should be chosen that is approximately 12-20° C. below the calculated Tₘ of the hybrid under study). The temperature and salt conditions can often be determined empirically in preliminary experiments in which samples of genomic DNA immobilized on filters are hybridized to the probe of interest and then washed under conditions of different stringencies.

6. To minimize background problems, it is best to hybridize for the shortest possible time using the minimum amount of probe. For Southern hybridization of mammalian genomic DNA where each specimen to be tested contains 10 μg of DNA, 10-20 ng/ml radiolabeled probe (sp. act.=10⁹ cpm/μg or greater) should be used and hybridization should be carried out for 12-16 hours at 68° C. in aqueous solution or for 24 hours at 42° C. in 50% formamide. For Southern hybridization of fragments of cloned DNA where each band of the restriction digest contains 10 ng of DNA or more, much less probe is required. Typically, hybridization is carried out for 6-8 hours using 1-2 ng/ml radiolabeled probe (sp. act.=10⁹ cpm/μg or greater).

The entire contents of all references cited above are hereby incorporated by reference.

While the present invention has been described in some detail for purposes of clarity and understanding, one skilled in the art will appreciate that various changes in form and detail can be made without departing from the true scope of the invention.

VII. Correlation of EST and Clone Identifiers

The EST sequences of the present invention are identified herein by EST Identifiers and SEQ ID NO, and deposits containing such clones have been submitted to the American Type Culture Collection (Rockville, Md. USA) as hereinabove indicated. All deposits have been made in accordance with the Budapest Treaty, and in full compliance with 37 CFR 1.801 et seq.

The Sequence Listing of this application is also identically provided in a computer readable format.

What is claimed is:

1. An isolated DNA sequence comprising DNA having at least a 95% identity to a DNA sequence selected from SEQ ID NOS:1-12483.

2. An isolated sequence comprising RNA corresponding to any of the DNA sequences or fragments of claim 1.

3. An isolated DNA sequence comprising a DNA sequence identical to a DNA sequence contained in and isolatable from ATCC Deposit No. 75916 by hybridization under stringent conditions with a DNA sequence of claim 1.

4. An isolated RNA sequence comprising RNA corresponding to any of the DNA sequences of claim 3.

5. An isolated DNA sequence containing at least the polypeptide coding region of a human gene, said human gene including a DNA sequence of claim 1.

6. An isolated DNA sequence comprising at least the polypeptide coding region of a human gene which contains a DNA sequence of claim 3.

7. The isolated DNA sequence of claim 6 which expresses a human protein when in a suitable expression system.

8. An expression vehicle comprising the DNA sequence of claim 1.

9. An expression vehicle comprising the DNA sequence of claim 3.

10. An expression vehicle comprising the DNA sequence of claim 5.

11. An expression vehicle comprising the DNA sequence of claim 7.

12. A polypeptide encoded by the DNA sequence of claim 5 and active fragments, derivatives and functional analogs thereof.

13. A polypeptide encoded by the DNA sequence of claim 6 and active fragments, derivatives and functional analogs thereof.

14. The isolated DNA sequence of claim 1 wherein the DNA sequence has at least a 97% identity to a DNA sequence selected from SEQ ID Nos. 1-4161.

15. The isolated DNA sequence of claim 1 wherein the DNA sequence has at least a 97% identity to a DNA sequence selected from SEQ ID Nos. 4162-8322.

16. The isolated DNA sequence of claim 1 wherein the DNA sequence has at least a 97% identity to a DNA sequence selected from SEQ ID Nos. 8323-12483.

17. A process for producing a polypeptide comprising:
expressing a polypeptide by use of DNA of claim 5.

18. An isolated DNA sequence encoding the same polypeptide as the DNA of claim 5.

19. An isolated DNA sequence encoding the same polypeptide as the DNA of claim 1.

20. An antibody against a polypeptide of claim 12.

21. A mixture of DNA sequences, said mixture containing at least thirty different DNA sequences of claim 1.

22. Cells engineered with DNA of claim 5.

23. A process for producing cells for expressing a polypeptide comprising:
genetically engineering cells with DNA of claim 5.

24. An isolated DNA sequence comprising a fragment of DNA having a sequence selected from SEQ ID Nos. 1-12483, wherein said fragment comprises at least 30 sequential bases of said sequence.

25. The isolated DNA of claim 1, wherein said DNA is identical to a DNA sequence selected from SEQ. ID Nos. 1-12483.

26. An isolated DNA sequence containing at least the coding region of a human gene, said human gene including a DNA sequence of claim 25.

27. An isolated DNA containing at least the polypeptide coding portion of a human gene, which isolate DNA is hybridizable to the DNA contained in a clone selected from the group consisting of the clones identified in Table 2.
